(19) United States
(12) Patent Application Publication
(10) Pub. No.: US 2025/0204838 A1
(43) Pub. Date: Jun. 26, 2025

(54) SYSTEM AND METHOD FOR DETECTION AND/OR PREDICTION OF ABNORMAL NEURAL ACTIVITY AND ASSOCIATED SUPPRESSION MEASURES

(71) Applicant: NEUROHELP LTD, Rehovot (IL)

(72) Inventors: Oren SHRIKI, Rehovot (IL); Shay BEN-SASSON, Even-Yehuda (IL)

(21) Appl. No.: 18/848,568

(22) PCT Filed: Mar. 14, 2023
(86) PCT No.: PCT/IL2023/050266

§ 371 Date: Sep. 19, 2024

Related U.S. Application Data
(60) Provisional application No. 63/322,650, filed on Mar. 23, 2022.

(51) Int. Cl. A61B 5/369; A61B 5/00; G16H 50/20; G16H 50/30

(52) U.S. Cl. CPC A61B 5/369; A61B 5/4848; G16H 50/20; G16H 50/30

(57) ABSTRACT

A system and method for detecting or predicting a given instance of an abnormal neural activity in a brain of a monitored person is provided. Features are extracted based on one or more neural signals that are indicative of given neural activity in the brain during a given time interval. At least some of the features are extracted based on neuronal avalanches. The given instance is detected or predicted based on the extracted features. There is also provided a system and method for verifying a prediction of a given instance of an abnormal neural activity, a system and method for automatically assisting a monitored person responsive to detection or prediction of a given instance of an abnormal neural activity, and a system and method for evaluating an effectiveness of a treatment plan for suppressing an onset of an abnormal neural activity.

### TECHNICAL FIELD

The invention relates to a system and method for detection and/or prediction of abnormal neural activity and associated suppression measures.

### BACKGROUND

A substantial number of persons who suffer from neural diseases live under constant fear of undergoing a neural disturbance since these persons do not know when they will be affected by a neural disturbance. For example, there are persons who suffer from epilepsy who live under constant fear of an impending epileptic seizure. An object of the present disclosure is to enable persons who suffer from neural diseases to anticipate that they will undergo a neural disturbance, thereby enabling these persons to be better prepared for the neural disturbance. An additional object of the present disclosure is to provide suppression measures for suppressing an expected or potential neural disturbance.

References considered to be relevant as background to the presently disclosed subject matter are listed below. Acknowledgement of the references herein is not to be inferred as meaning that these are in any way relevant to the patentability of the presently disclosed subject matter.

U.S. Pat. No. 8,548,786 (“Plenz”), published on Oct. 1, 2013, discloses a method and system for determining a cognitive enhancement and/or anti-epileptic effect comprising: detecting synchronized neuronal activity in neuronal tissue, monitoring spreading of the synchronized neuronal activity, determining a parameter indicative of the closeness of the synchronized neuronal activity to the critical state and comparing the parameter to a predetermined value.

International Patent Application Publication No. 2015/023929 (“Plenz et al.”), published on Feb. 19, 2015, discloses a method of continuously monitoring neuronal avalanches in a subject comprising (a) determining a deviation in avalanche exponent or branching parameter (σ) from a predetermined value at rest, wherein the pre-determined value of a is a slope of a size distribution of the synchronized neuronal activity and the predetermined value is −3/2 and the pre-determined value of a is a ratio of successively propagated synchronized neuronal activity and the predetermined value is 1; and (b) repeating step (a) one or more times to continuously monitor neuronal avalanches in a subject. Methods of determining or monitoring the degree of sleep deprivation in a subject, methods of identifying subjects that are susceptible to a sleep disorder and methods of diagnosing a sleep disorder in a subject are disclosed.

U.S. Pat. No. 10,172,567 (“Moxon et al.”), published on Jan. 8, 2019, describes systems and methods for predicting and detecting a seizure in a subject. The methods comprise measuring interneuron synchrony in terms of the coherence between interneuron action potentials and local field potential oscillations. In one embodiment, the detection of specific patterns of coherence, correlation and firing rate of interneurons predicts upcoming seizures.

U.S. Patent Application Publication No. 2010/0069776 (“Greger et al.”), published on Mar. 18, 2010, provides for the detection and monitoring of multiple micro-scale neurological signals indicative of neurological state, neurological activity, and/or neuropathology. By examining such micro-scale neurological signals, a care provider may make more accurate differential diagnoses, identify the most efficacious treatment strategy, and/or track the efficacy of treatment. In some embodiments, analysis of micro-scale electrophysiological signals can be used in the diagnosis, treatment decisions, and monitoring of several neurological disorders, e.g. epilepsy, movement disorders, and psychiatric disorders. In some embodiments, different cortical areas can be mapped, for example, to define boundaries between healthy and/or pathological neural tissue.

### GENERAL DESCRIPTION

In accordance with a first aspect of the presently disclosed subject matter, there is provided a method for detecting or predicting a given instance of an abnormal neural activity in a brain of a monitored person, the method comprising: extracting a plurality of features based on one or more neural signals that are indicative of given neural activity in the brain during a given time interval, wherein given features of the features are extracted based on neuronal avalanches, each avalanche of the avalanches being one or more consecutive sub-periods of distinct sub-periods within the given time interval in which one or more events associated with one or more of the neural signals are detected; and detecting the given instance or predicting the given instance within a given time duration of the given time interval, based on the plurality of features.

In some cases, one or more of the events are associated with a peak amplitude in a respective neural signal of the neural signals that is greater than or equal to a threshold.

In some cases, the given features include one or more inter-avalanche features that are extracted based on durations of inter-avalanche intervals between consecutive avalanches of the avalanches.

In some cases, the inter-avalanche features include one or more distribution-based features that are extracted based on an inter-avalanche distribution of the durations of the inter-avalanche intervals over the given time interval.

In some cases, the inter-avalanche distribution comprises: (a) a first regime for the inter-avalanche intervals that are of a short duration, the first regime being characterized by a first power law having a first exponent; (b) a second regime for the inter-avalanche intervals that are of a long duration, longer than the short duration, the second regime being characterized by a second power law having a second exponent, different than the first exponent; and (c) a transition region between the first regime and the second regime; and wherein the distribution-based features include at least one of: a first estimate of the first exponent; a second estimate of the second exponent; a third estimate of a crossover point between the first regime and the second regime, being an inter-avalanche interval duration within the transition region; a first deviation of the inter-avalanche intervals that are of the short duration from the first power law; or a second deviation of the inter-avalanche intervals that are of the long duration from the second power law.

In some cases, the given features include one or more multi-scale criticality features that are extracted by analyzing one or more basic features that are associated with the avalanches for different divisions of the given time interval into the distinct sub-periods.

In some cases, for one or more of the basic features, the multi-scale criticality features include an offset and a slope for a linear model that indicates a dependence of the respective basic feature on the different divisions.

In some cases, for one or more pairs of the basic features including a first basic feature and a second basic feature, the multi-scale criticality features include an offset and a slope for a linear model that indicates a dependence of a relationship between the first basic feature and the second basic feature of the respective pair on the different divisions.

In some cases, one of the basic features is an estimate of a branching parameter for the avalanches.

In some cases, a respective size of each avalanche of the avalanches is defined by a number of the events that are associated with the respective avalanche, wherein a regime of a size distribution of sizes of the avalanches over the given time interval is characterized by a power law having an exponent, wherein the basic features include at least one of: (a) an estimate of the exponent, (b) an exponential cut-off of the size distribution or (c) a deviation of the sizes from the power law.

In some cases, a regime of a duration distribution of durations of the avalanches over the given time interval is characterized by a power law having an exponent, wherein one of the basic features is an estimate of the exponent.

In some cases, for a plurality of durations of the avalanches over the given time interval, a linear model indicates a dependence of a mean size of the avalanches for each duration of the durations on the respective duration, the linear model being characterized by a power law having an exponent, wherein one of the basic features is an estimate of the exponent.

In some cases, an inter-avalanche distribution of durations of inter-avalanche intervals between consecutive avalanches of the avalanches over the given time interval comprises: (a) a first regime for the inter-avalanche intervals that are of a short duration, the first regime being characterized by a first power law having a first exponent; (b) a second regime for the inter-avalanche intervals that are of a long duration, longer than the short duration, the second regime being characterized by a second power law having a second exponent; and (c) a transition region between the first regime and the second regime; wherein the basic features include at least one of: a first estimate of the first exponent, a second estimate of the second exponent, or a third estimate of a crossover point between the first regime and the second regime, being an inter-avalanche interval duration within the transition region.

In some cases, a respective size of each avalanche of the avalanches is defined by a number of the events that are associated with the respective avalanche; wherein the avalanches consist of main avalanches and secondary avalanches, the main avalanches being the avalanches of a first size greater than or equal to a size threshold; and wherein the given features include one or more avalanche features that are extracted by analyzing a secondary avalanche rate distribution representing rates of secondary avalanches as a function of time that has elapsed since a preceding main avalanche of the main avalanches immediately preceding the secondary avalanches.

In some cases, the secondary avalanche rate distribution includes a regime that is characterized by a power law having an exponent, wherein the avalanche features include at least one of: an estimate of the exponent; or a deviation of the rates of the secondary avalanches fitted to the power law from the power law.

In some cases, a respective size of each avalanche of the avalanches is defined by a number of the events that are associated with the respective avalanche; wherein the given features include one or more additional avalanche features that are extracted by analyzing a function that estimates a relation between: (a) a difference in a size between consecutive avalanches of the avalanches and (b) a duration of an inter-avalanche interval between the consecutive avalanches.

In some cases, the additional avalanche features include: a value of a duration of the inter-avalanche interval at which a result of the function transitions from a negative value to a positive value or vice versa.

In some cases, at least one of the additional avalanche features is extracted by analyzing a shape of the function.

In some cases, in response to detecting or predicting the given instance, the method further comprises: automatically performing one or more actions.

In some cases, the actions include providing an alert.

In some cases, the actions include providing a treatment plan for the monitored person for suppressing the given instance or subsequent instances of the abnormal neural activity, the subsequent instances being subsequent to the given instance.

In some cases, the abnormal neural activity is an epileptic seizure.

In some cases, at least one given model analyzes the plurality of features to detect or predict the given instance.

In some cases, the given model is a Machine Learning (ML) or Deep Learning (DL) model.

In some cases, the abnormal neural activity is an epileptic seizure, and the given model provides one or more characteristics associated with the given instance upon detecting or predicting the given instance.

In some cases, the one or more characteristics include at least one of: a type of the epileptic seizure, one or more spatial characteristics associated with the epileptic seizure, a duration of the epileptic seizure, or a sub-duration within the given time duration in which the epileptic seizure is predicted to occur.

In some cases, the method further comprises: generating a new training record including the plurality of features and at least one label, the label being indicative of whether the given instance occurred during the given time interval or within the given time duration of the given time interval; updating a set of training records for training one or more models to include the new training record, giving rise to an updated set of training records for the one or more models; and updating the one or more models based on the updated set of training records.

In some cases, the one or more models include the given model.

In accordance with a second aspect of the presently disclosed subject matter, there is provided a method for verifying a given prediction, the method comprising: performing the given prediction, the given prediction predicting an onset of a given instance of an abnormal neural activity in a brain of a monitored person within a given time duration of a given time interval, based on given neural data that is indicative of given neural activity in the brain during the given time interval; performing one or more subsequent predictions, each subsequent prediction of the subsequent predictions being performed for predicting the onset of the given instance within a second given time duration of the respective subsequent prediction, based on subsequent neural data that is indicative of subsequent neural activity in the brain during a respective subsequent time interval of one or more subsequent time intervals, subsequent to the given time interval; and performing a verification of the given prediction, based on results of the subsequent predictions.

In some cases, the given prediction is verified upon one or more of the subsequent predictions predicting the onset of the given instance within the second given time duration of the respective subsequent prediction.

In some cases, the given prediction is verified upon at least a given percentage of the subsequent predictions predicting the onset of the given instance within the second given time duration of the respective subsequent prediction.

In some cases, the given prediction is verified upon at least two successive predictions of the subsequent predictions predicting the onset of the given instance within the second given time duration of the respective successive prediction, wherein the successive predictions are performed for successive time intervals of the subsequent time intervals.

In some cases, the given prediction is verified upon at least one subsequent prediction of the subsequent predictions that is performed for at least one successive time interval of the subsequent time intervals that is successive to the given time interval predicting the onset of the given instance within the second given time duration of the respective subsequent prediction.

In some cases, in response to verifying the given prediction, the method further comprises: performing one or more actions.

In some cases, the actions include providing an alert.

In some cases, the actions further include providing a confidence level, the confidence level being indicative of a probability of the onset of the given instance within the given time duration of the given time interval, and being based on a result of at least one of the subsequent predictions.

In some cases, the confidence level is based on a result of the given prediction and the subsequent predictions.

In some cases, in response to verifying the given prediction, the method further comprises: performing one or more additional predictions, each additional prediction of the additional predictions being performed for predicting the onset of the given instance within a third given time duration of the respective additional prediction, based on additional neural data that is indicative of additional neural activity in the brain during a respective additional time interval subsequent to the subsequent time intervals; and updating the confidence level, based on the additional predictions.

In some cases, the method further comprises: canceling the alert, based on the updated confidence level.

In some cases, the actions include providing a treatment plan for the monitored person for suppressing the given instance or subsequent instances of the abnormal neural activity, the subsequent instances being subsequent to the given instance.

In some cases, the method further comprises: providing, responsive to the given prediction, (a) an alert and (b) a confidence level that is indicative of a result of the given prediction.

In some cases, the method further comprises: updating the confidence level, based on the subsequent predictions.

In some cases, the verification of the given prediction is based on the updated confidence level.

In some cases, upon the given prediction being unverified based on the updated confidence level, the method further comprises: canceling the alert.

In some cases, the abnormal neural activity is an epileptic seizure.

In accordance with a third aspect of the presently disclosed subject matter, there is provided a method for automatically assisting a monitored person, the method comprising: detecting a given instance of an abnormal neural activity in a brain of the monitored person or predicting, in a given prediction, an onset of the given instance within a given time duration of a given time interval, based on given neural data that is indicative of given neural activity in the brain during the given time interval; and responsive to the detecting or the predicting, automatically assisting the monitored person.

In some cases, automatically assisting the monitored person includes providing a treatment plan for the monitored person for suppressing the given instance or subsequent instances of the abnormal neural activity, the subsequent instances being subsequent to the given instance.

In some cases, the treatment plan includes a new drug treatment or an optimization of an existing drug treatment for the monitored person.

In some cases, the treatment plan includes at least one biofeedback session or at least one neurofeedback session for the monitored person.

In some cases, the detecting or the predicting is based on one or more given features that are extracted based on neuronal avalanches, each avalanche of the avalanches being one or more consecutive sub-periods of distinct sub-periods within the predefined time interval in which one or more events associated with one or more neural signals are detected, the neural signals being indicative of the given neural activity in the brain during the given time interval; wherein, based on the biofeedback session or the neurofeedback session, at least one of the given features is modulated, thereby suppressing the given instance or the subsequent instances.

In some cases, the treatment plan includes vagal stimulation to be performed on the monitored person.

In some cases, automatically assisting the monitored person includes waking the monitored person or guiding the monitored person to a location that is more suitable for the monitored person to undergo the given instance.

In some cases, the abnormal neural activity is an epileptic seizure.

In accordance with a fourth aspect of the presently disclosed subject matter, there is provided a method for evaluating, during a given evaluation period, an effectiveness of a treatment plan for suppressing an onset of an abnormal neural activity in a brain of a monitored person, the given evaluation period being concurrent with or subsequent to a performance of the treatment plan, the method comprising: determining, for one or more given time intervals within the given evaluation period, a probability of an onset of an instance of the abnormal neural activity within a given time duration of the respective given time interval, based on given neural data that is indicative of given neural activity in the brain of the monitored person during the respective given time interval; and evaluating the effectiveness of the treatment plan, based on at least selected probabilities of the determined probabilities for the given time intervals.

In some cases, the method further comprises: calculating a prediction score, based on the selected probabilities; wherein the effectiveness of the treatment plan is evaluated based on the prediction score.

In some cases, the prediction score is calculated based on an average of the selected probabilities.

In some cases, the method further comprises: determining, for one or more second given time intervals within an earlier evaluation period preceding a performance of the treatment plan, a second probability of an onset of an earlier instance of the abnormal neural activity within a second given time duration of the respective second given time interval, based on earlier neural data that is indicative of earlier neural activity in the brain of the monitored person during the respective second predefined time interval; and calculating a second prediction score, based on at least selected second probabilities of the determined second probabilities; wherein the effectiveness of the treatment plan is evaluated based on the prediction score and the second prediction score.

In some cases, the second prediction score is calculated based on an average of the selected second probabilities.

In some cases, the treatment plan includes a new drug treatment or an optimization of an existing drug treatment for the monitored person.

In some cases, the treatment plan includes at least one biofeedback session or at least one neurofeedback session for the monitored person.

In some cases, the treatment plan includes vagal stimulation on the monitored person.

In some cases, the abnormal neural activity is an epileptic seizure.

In accordance with a fifth aspect of the presently disclosed subject matter, there is provided a system for detecting or predicting a given instance of an abnormal neural activity in a brain of a monitored person, the system comprising a processing circuitry configured to: extract a plurality of features based on one or more neural signals that are indicative of given neural activity in the brain during a given time interval, wherein given features of the features are extracted based on neuronal avalanches, each avalanche of the avalanches being one or more consecutive sub-periods of distinct sub-periods within the given time interval in which one or more events associated with one or more of the neural signals are detected; and detect the given instance or predict the given instance within a given time duration of the given time interval, based on the plurality of features.

In some cases, one or more of the events are associated with a peak amplitude in a respective neural signal of the neural signals that is greater than or equal to a threshold.

In some cases, the given features include one or more inter-avalanche features that are extracted based on durations of inter-avalanche intervals between consecutive avalanches of the avalanches.

In some cases, the inter-avalanche features include one or more distribution-based features that are extracted based on an inter-avalanche distribution of the durations of the inter-avalanche intervals over the given time interval.

In some cases, the inter-avalanche distribution comprises: (a) a first regime for the inter-avalanche intervals that are of a short duration, the first regime being characterized by a first power law having a first exponent; (b) a second regime for the inter-avalanche intervals that are of a long duration, longer than the short duration, the second regime being characterized by a second power law having a second exponent, different than the first exponent; and (c) a transition region between the first regime and the second regime; wherein the distribution-based features include at least one of: a first estimate of the first exponent; a second estimate of the second exponent; a third estimate of a crossover point between the first regime and the second regime, being an inter-avalanche interval duration within the transition region; a first deviation of the inter-avalanche intervals that are of the short duration from the first power law; or a second deviation of the inter-avalanche intervals that are of the long duration from the second power law.

In some cases, the given features include one or more multi-scale criticality features that are extracted by analyzing one or more basic features that are associated with the avalanches for different divisions of the given time interval into the distinct sub-periods.

In some cases, for one or more of the basic features, the multi-scale criticality features include an offset and a slope for a linear model that indicates a dependence of the respective basic feature on the different divisions.

In some cases, for one or more pairs of the basic features including a first basic feature and a second basic feature, the multi-scale criticality features include an offset and a slope for a linear model that indicates a dependence of a relationship between the first basic feature and the second basic feature of the respective pair on the different divisions.

In some cases, one of the basic features is an estimate of a branching parameter for the avalanches.

In some cases, a respective size of each avalanche of the avalanches is defined by a number of the events that are associated with the respective avalanche, wherein a regime of a size distribution of sizes of the avalanches over the given time interval is characterized by a power law having an exponent, and wherein the basic features include at least one of: (a) an estimate of the exponent, (b) an exponential cut-off of the size distribution or (c) a deviation of the sizes from the power law.

In some cases, a regime of a duration distribution of durations of the avalanches over the given time interval is characterized by a power law having an exponent, wherein one of the basic features is an estimate of the exponent.

In some cases, for a plurality of durations of the avalanches over the given time interval, a linear model indicates a dependence of a mean size of the avalanches for each duration of the durations on the respective duration, the linear model being characterized by a power law having an exponent, wherein one of the basic features is an estimate of the exponent.

In some cases, an inter-avalanche distribution of durations of inter-avalanche intervals between consecutive avalanches of the avalanches over the given time interval comprises: (a) a first regime for the inter-avalanche intervals that are of a short duration, the first regime being characterized by a first power law having a first exponent; (b) a second regime for the inter-avalanche intervals that are of a long duration, longer than the short duration, the second regime being characterized by a second power law having a second exponent; and (c) a transition region between the first regime and the second regime; wherein the basic features include at least one of: a first estimate of the first exponent, a second estimate of the second exponent, or a third estimate of a crossover point between the first regime and the second regime, being an inter-avalanche interval duration within the transition region.

In some cases, a respective size of each avalanche of the avalanches is defined by a number of the events that are associated with the respective avalanche; wherein the avalanches consist of main avalanches and secondary avalanches, the main avalanches being the avalanches of a first size greater than or equal to a size threshold; and wherein the given features include one or more avalanche features that are extracted by analyzing a secondary avalanche rate distribution representing rates of secondary avalanches as a function of time that has elapsed since a preceding main avalanche of the main avalanches immediately preceding the secondary avalanches.

In some cases, the secondary avalanche rate distribution includes a regime that is characterized by a power law having an exponent, wherein the avalanche features include at least one of: an estimate of the exponent; or a deviation of the rates of the secondary avalanches fitted to the power law from the power law.

In some cases, a respective size of each avalanche of the avalanches is defined by a number of the events that are associated with the respective avalanche; wherein the given features include one or more additional avalanche features that are extracted by analyzing a function that estimates a relation between: (a) a difference in a size between consecutive avalanches of the avalanches and (b) a duration of an inter-avalanche interval between the consecutive avalanches.

In some cases, the additional avalanche features include: a value of a duration of the inter-avalanche interval at which a result of the function transitions from a negative value to a positive value or vice versa.

In some cases, at least one of the additional avalanche features is extracted by analyzing a shape of the function.

In some cases, in response to detecting or predicting the given instance, the processing circuitry is further configured to: automatically perform one or more actions.

In some cases, the actions include providing an alert.

In some cases, the actions include providing a treatment plan for the monitored person for suppressing the given instance or subsequent instances of the abnormal neural activity, the subsequent instances being subsequent to the given instance.

In some cases, the abnormal neural activity is an epileptic seizure.

In some cases, at least one given model analyzes the plurality of features to detect or predict the given instance.

In some cases, the given model is a Machine Learning (ML) or Deep Learning (DL) model.

In some cases, the abnormal neural activity is an epileptic seizure, and the given model provides one or more characteristics associated with the given instance upon detecting or predicting the given instance.

In some cases, the one or more characteristics include at least one of: a type of the epileptic seizure, one or more spatial characteristics associated with the epileptic seizure, a duration of the epileptic seizure, or a sub-duration within the given time duration in which the epileptic seizure is predicted to occur.

In some cases, the processing circuitry is further configured to: generate a new training record including the plurality of features and at least one label, the label being indicative of whether the given instance occurred during the given time interval or within the given time duration of the given time interval; update a set of training records for training one or more models to include the new training record, giving rise to an updated set of training records for the one or more models; and update the one or more models based on the updated set of training records.

In some cases, the one or more models include the given model.

In accordance with a sixth aspect of the presently disclosed subject matter, there is provided a system for verifying a given prediction, the system comprising a processing circuitry configured to: perform the given prediction, the given prediction predicting an onset of a given instance of an abnormal neural activity in a brain of a monitored person within a given time duration of a given time interval, based on given neural data that is indicative of given neural activity in the brain during the given time interval; perform one or more subsequent predictions, each subsequent prediction of the subsequent predictions being performed for predicting the onset of the given instance within a second given time duration of the respective subsequent prediction, based on subsequent neural data that is indicative of subsequent neural activity in the brain during a respective subsequent time interval of one or more subsequent time intervals, subsequent to the given time interval; and perform a verification of the given prediction, based on results of the subsequent predictions.

In some cases, the given prediction is verified upon one or more of the subsequent predictions predicting the onset of the given instance within the second given time duration of the respective subsequent prediction.

In some cases, the given prediction is verified upon at least a given percentage of the subsequent predictions predicting the onset of the given instance within the second given time duration of the respective subsequent prediction.

In some cases, the given prediction is verified upon at least two successive predictions of the subsequent predictions predicting the onset of the given instance within the second given time duration of the respective successive prediction, wherein the successive predictions are performed for successive time intervals of the subsequent time intervals.

In some cases, the given prediction is verified upon at least one subsequent prediction of the subsequent predictions that is performed for at least one successive time interval of the subsequent time intervals that is successive to the given time interval predicting the onset of the given instance within the second given time duration of the respective subsequent prediction.

In some cases, in response to verifying the given prediction, the processing circuitry is further configured to: perform one or more actions.

In some cases, the actions include providing an alert.

In some cases, the actions further include providing a confidence level, the confidence level being indicative of a probability of the onset of the given instance within the given time duration of the given time interval, and being based on a result of at least one of the subsequent predictions.

In some cases, the confidence level is based on a result of the given prediction and the subsequent predictions.

In some cases, in response to verifying the given prediction, the processing circuitry is further configured to: perform one or more additional predictions, each additional prediction of the additional predictions being performed for predicting the onset of the given instance within a third given time duration of the respective additional prediction, based on additional neural data that is indicative of additional neural activity in the brain during a respective additional time interval subsequent to the subsequent time intervals; and update the confidence level, based on the additional predictions.

In some cases, the processing circuitry is further configured to: cancel the alert, based on the updated confidence level.

In some cases, the actions include providing a treatment plan for the monitored person for suppressing the given instance or subsequent instances of the abnormal neural activity, the subsequent instances being subsequent to the given instance.

In some cases, the processing circuitry is further configured to: provide, responsive to the given prediction, (a) an alert and (b) a confidence level that is indicative of a result of the given prediction.

In some cases, the processing circuitry is further configured to: update the confidence level, based on the subsequent predictions.

In some cases, the verification of the given prediction is based on the updated confidence level.

In some cases, upon the given prediction being unverified based on the updated confidence level, the processing circuitry is further configured to: cancel the alert.

In some cases, the abnormal neural activity is an epileptic seizure.

In accordance with a seventh aspect of the presently disclosed subject matter, there is provided a system for automatically assisting a monitored person, the system comprising a processing circuitry configured to: detect a given instance of an abnormal neural activity in a brain of the monitored person or predict, in a given prediction, an onset of the given instance within a given time duration of a given time interval, based on given neural data that is indicative of given neural activity in the brain during the given time interval; and responsive to the detect or the predict, automatically assist the monitored person.

In some cases, automatically assisting the monitored person includes providing a treatment plan for the monitored person for suppressing the given instance or subsequent instances of the abnormal neural activity, the subsequent instances being subsequent to the given instance.

In some cases, the treatment plan includes a new drug treatment or an optimization of an existing drug treatment for the monitored person.

In some cases, the treatment plan includes at least one biofeedback session or at least one neurofeedback session for the monitored person.

In some cases, the detect or the predict is based on one or more given features that are extracted based on neuronal avalanches, each avalanche of the avalanches being one or more consecutive sub-periods of distinct sub-periods within the predefined time interval in which one or more events associated with one or more neural signals are detected, the neural signals being indicative of the given neural activity in the brain during the given time interval; and wherein, based on the biofeedback session or the neurofeedback session, at least one of the given features is modulated, thereby suppressing the given instance or the subsequent instances.

In some cases, the treatment plan includes vagal stimulation to be performed on the monitored person.

In some cases, automatically assisting the monitored person includes waking the monitored person or guiding the monitored person to a location that is more suitable for the monitored person to undergo the given instance.

In some cases, the abnormal neural activity is an epileptic seizure.

In accordance with an eighth aspect of the presently disclosed subject matter, there is provided a system for evaluating, during a given evaluation period, an effectiveness of a treatment plan for suppressing an onset of an abnormal neural activity in a brain of a monitored person, the given evaluation period being concurrent with or subsequent to a performance of the treatment plan, the system comprising a processing circuitry configured to: determine, for one or more given time intervals within the given evaluation period, a probability of an onset of an instance of the abnormal neural activity within a given time duration of the respective given time interval, based on given neural data that is indicative of given neural activity in the brain of the monitored person during the respective given time interval; and evaluate the effectiveness of the treatment plan, based on at least selected probabilities of the determined probabilities for the given time intervals.

In some cases, the processing circuitry is further configured to: calculate a prediction score, based on the selected probabilities; wherein the effectiveness of the treatment plan is evaluated based on the prediction score.

In some cases, the prediction score is calculated based on an average of the selected probabilities.

In some cases, the processing circuitry is further configured to: determine, for one or more second given time intervals within an earlier evaluation period preceding a performance of the treatment plan, a second probability of an onset of an earlier instance of the abnormal neural activity within a second given time duration of the respective second given time interval, based on earlier neural data that is indicative of earlier neural activity in the brain of the monitored person during the respective second predefined time interval; and calculate a second prediction score, based on at least selected second probabilities of the determined second probabilities; wherein the effectiveness of the treatment plan is evaluated based on the prediction score and the second prediction score.

In some cases, the second prediction score is calculated based on an average of the selected second probabilities.

In some cases, the treatment plan includes a new drug treatment or an optimization of an existing drug treatment for the monitored person.

In some cases, the treatment plan includes at least one biofeedback session or at least one neurofeedback session for the monitored person.

In some cases, the treatment plan includes vagal stimulation on the monitored person.

In some cases, the abnormal neural activity is an epileptic seizure.

In accordance with a ninth aspect of the presently disclosed subject matter, there is provided a non-transitory computer readable storage medium having computer readable program code embodied therewith, the computer readable program code, executable by processing circuitry of a computer to perform a method for detecting or predicting a given instance of an abnormal neural activity in a brain of a monitored person, the method comprising: extracting a plurality of features based on one or more neural signals that are indicative of given neural activity in the brain during a given time interval, wherein given features of the features are extracted based on neuronal avalanches, each avalanche of the avalanches being one or more consecutive sub-periods of distinct sub-periods within the given time interval in which one or more events associated with one or more of the neural signals are detected; and detecting the given instance or predicting the given instance within a given time duration of the given time interval, based on the plurality of features.

In accordance with a tenth aspect of the presently disclosed subject matter, there is provided a non-transitory computer readable storage medium having computer readable program code embodied therewith, the computer readable program code, executable by processing circuitry of a computer to perform a method for verifying a given prediction, the method comprising: performing the given prediction, the given prediction predicting an onset of a given instance of an abnormal neural activity in a brain of a monitored person within a given time duration of a given time interval, based on given neural data that is indicative of given neural activity in the brain during the given time interval; performing one or more subsequent predictions, each subsequent prediction of the subsequent predictions being performed for predicting the onset of the given instance within a second given time duration of the respective subsequent prediction, based on subsequent neural data that is indicative of subsequent neural activity in the brain during a respective subsequent time interval of one or more subsequent time intervals, subsequent to the given time interval; and performing a verification of the given prediction, based on results of the subsequent predictions.

In accordance with an eleventh aspect of the presently disclosed subject matter, there is provided a non-transitory computer readable storage medium having computer readable program code embodied therewith, the computer readable program code, executable by processing circuitry of a computer to perform a method for automatically assisting a monitored person, the method comprising: detecting a given instance of an abnormal neural activity in a brain of the monitored person or predicting, in a given prediction, an onset of the given instance within a given time duration of a given time interval, based on given neural data that is indicative of given neural activity in the brain during the given time interval; and responsive to the detecting or the predicting, automatically assisting the monitored person.

In accordance with a twelfth aspect of the presently disclosed subject matter, there is provided a non-transitory computer readable storage medium having computer readable program code embodied therewith, the computer readable program code, executable by processing circuitry of a computer to perform a method for evaluating, during a given evaluation period, an effectiveness of a treatment plan for suppressing an onset of an abnormal neural activity in a brain of a monitored person, the given evaluation period being concurrent with or subsequent to a performance of the treatment plan, the method comprising: determining, for one or more given time intervals within the given evaluation period, a probability of an onset of an instance of the abnormal neural activity within a given time duration of the respective given time interval, based on given neural data that is indicative of given neural activity in the brain of the monitored person during the respective given time interval; and evaluating the effectiveness of the treatment plan, based on at least selected probabilities of the determined probabilities for the given time intervals.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a block diagram schematically illustrating one example of an operation of an abnormal neural activity detection/prediction system, in accordance with the presently disclosed subject matter;

FIG. 2 is a schematic illustration of one example of a given neural signal of neural signals for a given time interval, in accordance with the presently disclosed subject matter;

FIG. 3A is a schematic illustration that illustrates an example of a first way for clustering events that are associated with neural signals into neuronal avalanches, in accordance with the presently disclosed subject matter;

FIG. 3B is a schematic illustration that illustrates an example of a second way for clustering events that are associated with the neural signals into neuronal avalanches, in accordance with the presently disclosed subject matter;

FIG. 4 is a block diagram schematically illustrating one example of an abnormal neural activity detection/prediction system, in accordance with the presently disclosed subject matter;

FIG. 5 is a flow diagram schematically illustrating one example of a sequence of operations for detecting or predicting a given instance of an abnormal neural activity in the brain of a monitored person, in accordance with the presently disclosed subject matter;

FIG. 6 is a graph that schematically illustrates one example of an inter-avalanche distribution of durations of inter-avalanche intervals over a given time interval, in accordance with the presently disclosed subject matter;

FIG. 7 is a graph that schematically illustrates one example of a secondary avalanche rate distribution of a normalized secondary avalanche rate as a function of time that has elapsed since a preceding main avalanche, in accordance with the presently disclosed subject matter;

FIG. 8 is a flow diagram schematically illustrating one example of a sequence of operations for verifying a given prediction predicting an onset of a given instance of an abnormal neural activity in a brain of a monitored person, in accordance with the presently disclosed subject matter;

FIG. 9 is a flow diagram schematically illustrating one example of a sequence of operations for automatically assisting a monitored person in response to a detection or prediction of a given instance of an abnormal neural activity in the brain of the monitored person, in accordance with the presently disclosed subject matter; and

FIG. 10 is a flow diagram schematically illustrating one example of a sequence of operations for evaluating, during a given evaluation period, an effectiveness of a treatment plan for suppressing an onset of an abnormal neural activity in a brain of a monitored person, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “extracting”, “detecting”, “predicting”, “analyzing”, “performing”, “providing”, “generating”, “updating”, “verifying”, “canceling”, “assisting”, “determining”, “evaluating”, “calculating” or the like, include actions and/or processes, including, inter alia, actions and/or processes of a computer, that manipulate and/or transform data into other data, said data represented as physical quantities, e.g. such as electronic quantities, and/or said data representing the physical objects. The terms “computer”, “processor”, “processing circuitry” and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field-programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

As used herein, the phrase “for example,” “an additional example”, “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

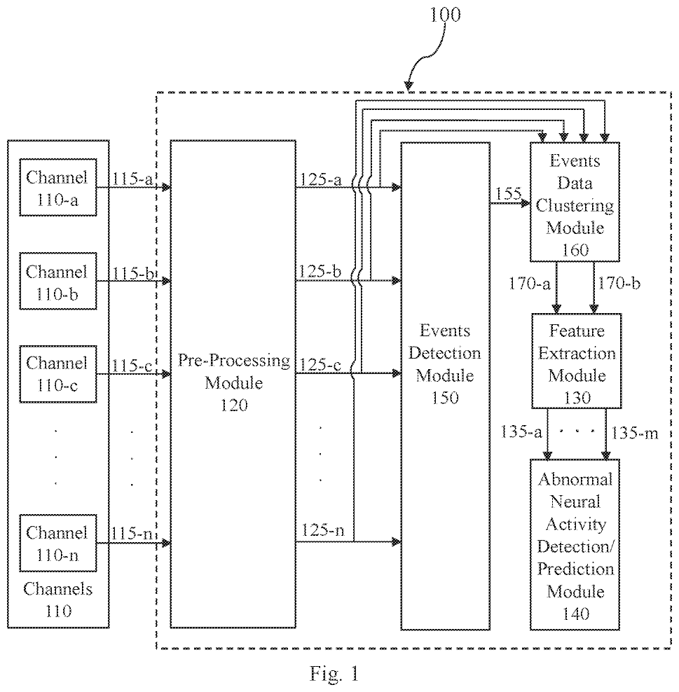

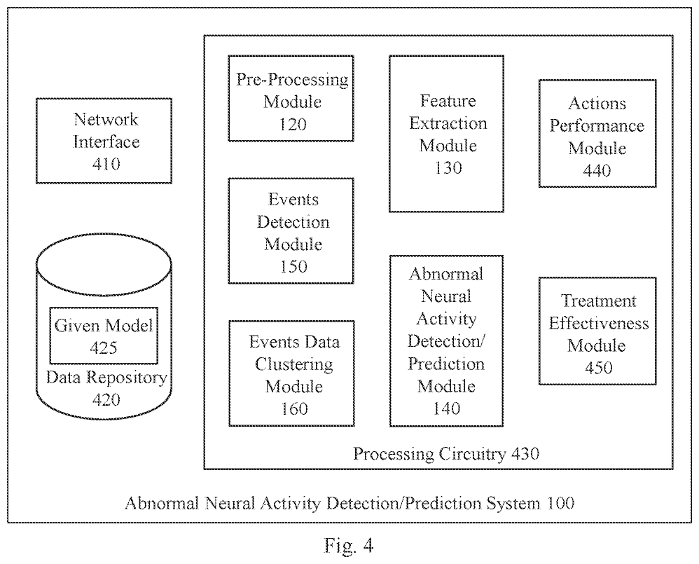

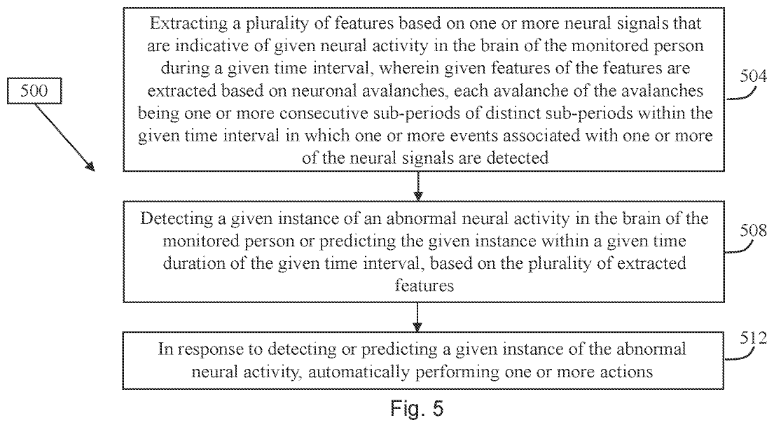

In embodiments of the presently disclosed subject matter, fewer, more and/or different stages than those shown in FIGS. 5 and 8 to 10 may be executed. FIGS. 1 and 4 illustrate a general schematic of the system architecture in accordance with embodiments of the presently disclosed subject matter. Each module in FIGS. 1 and 4 can be made up of any combination of software, hardware and/or firmware that performs the functions as defined and explained herein. The modules in FIGS. 1 and 4 may be centralized in one location or dispersed over more than one location. In other embodiments of the presently disclosed subject matter, the system may comprise fewer, more, and/or different modules than those shown in FIGS. 1 and 4.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Attention is now drawn to FIG. 1, a block diagram schematically illustrating one example of an operation of an abnormal neural activity detection/prediction system 100, in accordance with the presently disclosed subject matter.

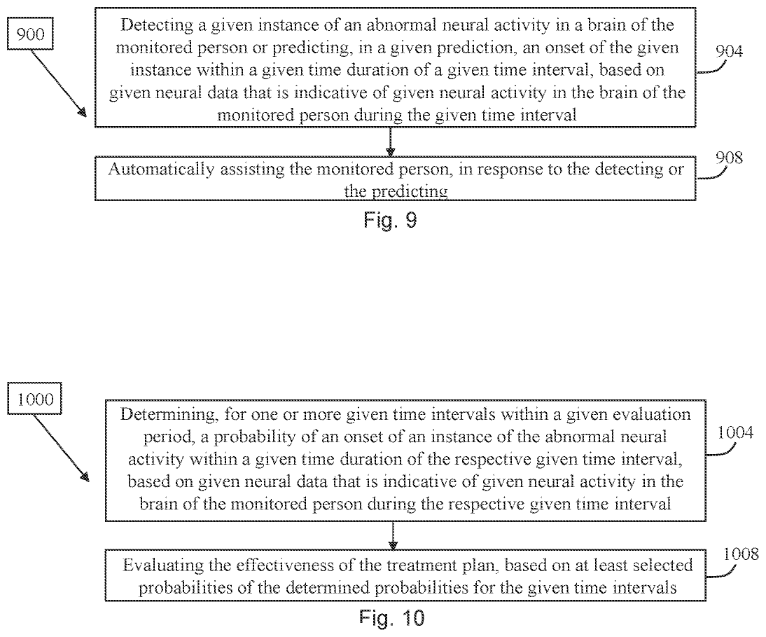

In accordance with the presently disclosed subject matter, an abnormal neural data detection/prediction system 100 can be configured to detect and/or predict instances of an abnormal neural activity in a brain of a monitored person, the abnormal neural activity being associated with a neurological disorder. This can enable the monitored person to be better prepared for an instance of the abnormal neural activity, as detailed further herein, inter alia with reference to FIGS. 5, 8 and 9. In some cases, abnormal neural data detection/prediction system 100 can further be configured to suppress a predicted instance of an abnormal neural activity, as detailed further herein, inter alia with reference to FIG. 9.

In some cases, the abnormal neural activity can be an epileptic seizure. However, the abnormal neural activity can also be, for example (non-limiting), one or more of the following: a migraine, an Alzheimer attack, a Parkinson's attack, a stroke, a multiple sclerosis attack, a psychiatric or psychosis episode, an episode of sleep deprivation or disorder, a disorder of consciousness (DOC) episode or a depressive episode.

In order to detect or predict an instance of the abnormal neural activity, neural data (e.g., 115-a, 115-b, 115-c, . . . 115-n) that is indicative of neural activity in the monitored person's brain is sampled from a plurality of channels 110 (e.g., 110-a, 110-b, 110-c, . . . , 110-n) over one or more time intervals. In some cases, at least one of the time intervals is between about 6 seconds to about 30 minutes. In some cases, at least one of the time intervals is between about 2 minutes to about 6 minutes. In some cases, all of the time intervals are of the same duration. In some cases, abnormal neural activity detection/prediction system 100 can be configured to sample the neural data (e.g., 115-a, 115-b, 115-c, . . . 115-n) from the channels 110, as illustrated in FIG. 1.

In some cases, the channels 110 can be electrodes, and the neural data (e.g., 115-a, 115-b, 115-c, . . . , 115-n) can be electroencephalogram (EEG) measurements obtained from the electrodes. By monitoring the neural activity in the brain of the monitored person using EEG recordings, the detection and/or prediction of abnormal neural activity in the brain of the monitored person can be carried out reliably, with ease, and relatively inexpensively.

In some cases, one or more of the channels 110 are surface electrodes that are placed on a surface of the head of the monitored person. Additionally, or alternatively, in some cases, one or more of the channels 110 are subcutaneous electrodes that are placed under the skin of the head of the monitored person. Additionally, or as a further alternative, in some cases, one or more of the channels 110 are implantable electrodes that are implanted in the head of the monitored person. In some cases, all of the channels 110 are either surface electrodes, subcutaneous electrodes or implantable electrodes. In some cases in which the channels 110 are electrodes that provide EEG measurements, the number of channels 110 can be: (a) between one and one hundred, (b) between five and twenty or (c) between six and ten.

In some cases, the channels 110 can be magnetometers or gradiometers, and the neural data (e.g., 115-a, 115-b, 115-c, . . . , 115-n) can be magnetoencephalogram (MEG) measurements obtained from the magnetometers or gradiometers. In some cases, the magnetometers or gradiometers can be placed in a helmet-shaped vacuum flask that covers at least part of the head of the monitored person. In some cases, the MEG system can be mobile.

For one or more (e.g., each) of the time intervals over which the neural data (e.g., 115-a, 115-b, 115-c, . . . , 115-n) is sampled, the neural data (e.g., 115-a, 115-b, 115-c, . . . , 115-n) is configured to be pre-processed, e.g. using a pre-processing module 120. In some cases, at least some of the pre-processing of the neural data (e.g., 115-a, 115-b, 115-c, . . . , 115-n) is performed by abnormal neural activity detection/prediction system 100, as illustrated in FIG. 1. In some cases, for one or more (e.g., each) of the time intervals over which the neural data (e.g., 115-a, 115-b, 115-c, . . . , 115-n) is sampled, the pre-processing of the neural data (e.g., 115-a, 115-b, 115-c, . . . , 115-n) for each channel of the channels 110 includes normalization of the neural data (e.g., 115-a, 115-b, 115-c, . . . , 115-n) for the respective channel. In some cases, the normalization of the neural data for a respective channel can be performed by calculating z-scores (i.e., standard scores) for the data samples of the neural data that is obtained from the respective channel (e.g., 110-a, 110-b, 110-c, . . . , 110-n). The pre-processing of the neural data (e.g., 115-a, 115-b, 115-c, . . . , 115-n) for any respective time interval gives rise to neural signals (e.g., 125-a, 125-b, 125-c, . . . , 125-n), each neural signal of the neural signals (e.g., 125-a, 125-b, 125-c, . . . , 125-n) being associated with a distinct channel of the channels 110.

In some cases, abnormal neural data detection/prediction system 100 can be configured, e.g. using feature extraction module 130, to extract, for one or more time intervals, a plurality of features (e.g., 135-a, . . . , 135-m) that are associated with the respective time interval. The plurality of features (e.g., 135-a, . . . , 135-m) that are associated with a respective time interval are extracted based on neural data (e.g., 115-a, 115-b, 115-c, . . . , 115-n) that is indicative of neural activity in the brain of the monitored person during the respective time interval. In some cases, at least some of these features can be extracted based on the neural signals (e.g., 125-a, 125-b, 125-c, . . . , 125-n) that are derived based on the neural data (e.g., 115-a, 115-b, 115-c, . . . , 115-n) associated with the respective time interval.

Abnormal neural data detection/prediction system 100 can be further configured, e.g. using abnormal neural data detection/prediction module 140, to detect a given instance of an abnormal neural activity in the brain of the monitored person or to predict the given instance within a given time duration of a given time interval, based on the plurality of features (e.g., 135-a, . . . , 135-m) associated with the given time interval that are extracted by the abnormal neural data detection/prediction system 100. In some cases, the detection or prediction of the given instance can also be based on features that are extracted by an external computing entity (not shown), external to abnormal neural data detection/prediction system 100. In some cases, the given time duration can be within a range of 5 minutes to 8 hours. In some cases, the given time duration can be within a range of 10 minutes to 4 hours (e.g., 10 minutes, 15 minutes, 20 minutes, 25 minutes, 30 minutes, 35 minutes, 40 minutes, 45 minutes, 50 minutes, 55 minutes, one hour, 75 minutes, 90 minutes, 105 minutes, two hours, two and a half hours, three hours, three and a half hours, four hours).

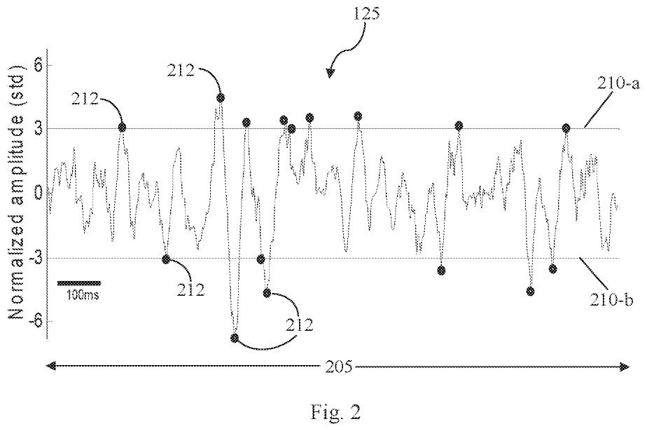

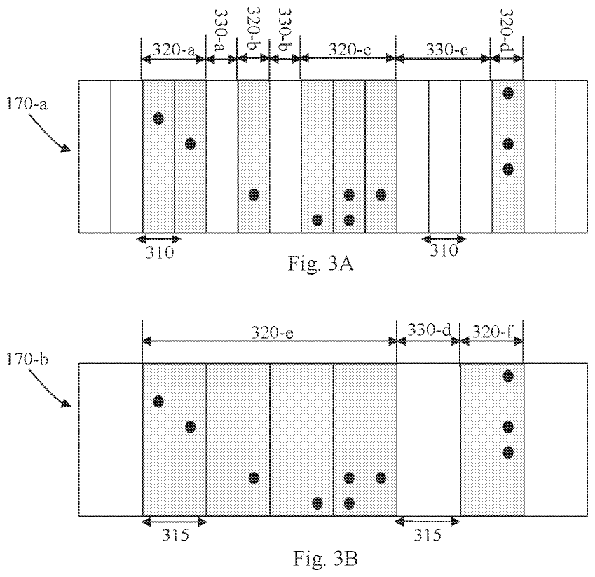

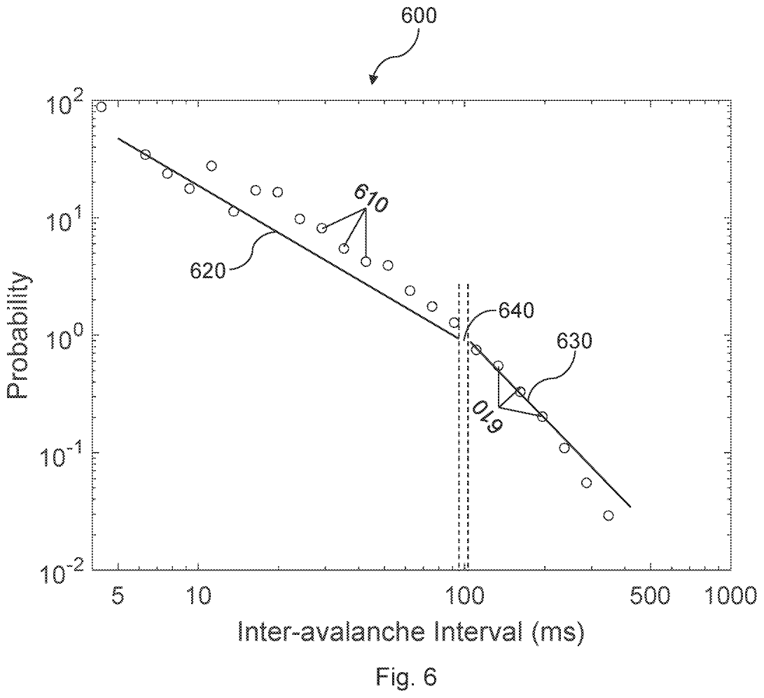

In some cases, the plurality of features (e.g., 135-a, . . . , 135-m) that are extracted by abnormal neural data detection/prediction system 100 for a respective time interval can include given features that are extracted based on neuronal avalanches associated with the respective time interval. The neuronal avalanches are spatiotemporal cascades of bursts of neuronal activity in the brain of the monitored person. Each avalanche of the neuronal avalanches is one or more consecutive sub-periods of distinct sub-periods within a respective time interval in which one or more events 155 are detected, as detailed further herein, inter alia with reference to FIGS. 3A and 3B. In some cases, one or more of the events 155 for a respective time interval can be associated with a peak amplitude in a respective neural signal of the neural signals (e.g., 125-a, 125-b, 125-c, . . . , 125-n) associated with the respective time interval that is greater than or equal to a threshold, as detailed further herein, inter alia with reference to FIG. 2. Additionally, or alternatively, in some cases, a template of an event can be defined, and one or more of the events 155 for a respective time interval can each be associated with a location in a respective neural signal of the neural signals (e.g., 125-a, 125-b, 125-c, . . . , 125-n) associated with the respective time interval that is highly correlated with the template. It is to be noted that, for the purposes of the present disclosure, each of the events 155 is indicative of an active channel of the channels 110, however the respective event is detected. In some cases, the given features that are extracted based on the neuronal avalanches can include certain features that are extracted based on inter-avalanche intervals between consecutive neuronal avalanches of the neuronal avalanches, as detailed further herein, inter alia with reference to FIGS. 5 and 6.

In some cases, in order to detect one or more of the events 155 for a given time interval, a threshold is applied to each of the neural signals (e.g., 125-a, 125-b, 125-c, . . . , 125-n) that is associated with the given time interval. The threshold that is applied to each of the neural signals (e.g., 125-a, 125-b, 125-c, . . . , 125-n) is applied to both positive values and negative values of the respective neural signal. In some cases, the threshold that is applied to each of the neural signals (e.g., 125-a, 125-b, 125-c, . . . , 125-n) can be a given multiple (an integer or a non-integer multiple) of a standard deviation of the respective neural signal. In some cases (non-limiting), the given multiple can be within the range of about 2.5 to about 4. In some cases, in contrast, the threshold that is applied to each of the neural signals (e.g., 125-a, 125-b, 125-c, . . . , 125-n) can be an absolute threshold. In some cases, abnormal neural activity detection/prediction system 100 can be configured, e.g. using events detection module 150, to detect the events 155 for any given time interval. It is to be further noted that for each event that is detected for a given time interval, a time of occurrence of the event is also retained, thereby enabling the clustering of events 155 that are associated with a given time interval into neuronal avalanches.

To explain how one or more of the events 155 for a given time interval are detected in accordance with one embodiment of the present disclosure, attention is briefly drawn to FIG. 2, a schematic illustration of one example of a given neural signal 125 of the neural signals (e.g., 125-a, 125-b, 125-c, . . . , 125-n) for the given time interval, in accordance with the presently disclosed subject matter.

In accordance with the presently disclosed subject matter, each neural signal of the neural signals (e.g., 125-a, 125-b, 125-c, . . . , 125-n), including, inter alia, given neural signal 125, can be generated, in some cases, by performing data normalization on corresponding neural data (e.g., 115-a, 115-b, 115-c, 115-d) that is obtained from a respective channel of the channels 110 over a given time interval 205, as illustrated in FIG. 2. In some cases, a given threshold (210-a, 210-b) can be applied to each of the neural signals (e.g., 125-a, 125-b, 125-c, . . . , 125-n) to detect one or more of the events 155 for the given time interval 205. That is, the threshold (210-a, 210-b) is determined identically for each of the neural signals (e.g., 125-a, 125-b, 125-c, . . . , 125-n). In some cases, the threshold (210-a, 210-b) can be a given multiple (an integer or a non-integer multiple) of a standard deviation of the respective neural signal. In some cases, the threshold (210-a, 210-b) can be three standard deviations (std) of the respective neural signal, as illustrated in FIG. 2, with respect to the given neural signal 125. In some cases, one or more of the events 155 that are associated with the given neural signal 125 is associated with a peak amplitude 212 in the given neural signal 125 that is greater than or equal to the threshold (210-a, 210-b). It is to be noted that the threshold (210-a, 210-b) of three std is provided for exemplary purposes only, and that the threshold can be another value, as detailed earlier herein.

The events that are associated with the neural signals (e.g., 125-a, 125-b, 125-c, . . . 125-n) can be clustered into neuronal avalanches, being spatiotemporal cascades of bursts of neuronal activity in the brain of the monitored person. In some cases, abnormal neural activity detection/prediction system 100 can be configured to extract given features based on the neuronal avalanches, e.g. using feature extraction module 130, and to detect or predict a given instance of an abnormal neural activity based on the given features, e.g. using abnormal neural activity detection/prediction module 140. In some cases, the events 155 can be clustered into neuronal avalanches in only one way by dividing a respective time interval (e.g., 205) that is associated with the neural signals (e.g., 125-a, 125-b, 125-c, . . . , 125-n) into distinct sub-periods of about equal duration in one manner. Alternatively, in some cases, the events 155 can be clustered into neuronal avalanches in two or more ways by dividing the respective time interval (e.g., 205) into distinct sub-periods of about equal duration in a corresponding two or more manners, as detailed further herein, inter alia with reference to FIGS. 3A and 3B.

In some cases, abnormal neural activity detection/prediction system 100 can be configured, e.g. using events data clustering module 160, to cluster the events 155 that are associated with a respective time interval (e.g., 205) into neuronal avalanches, as illustrated in FIG. 1. FIG. 1 illustrates the clustering of the events 155 into neuronal avalanches in two ways (170-a, 170-b); however, such a clustering of the events 155 into neuronal avalanches is only exemplary and non-limiting. It is to be noted that by clustering the events 155 into neuronal avalanches in two or more ways (e.g., 170-a, 170-b), multi-scale criticality features can be extracted for detecting or predicting a given instance of an abnormal neural activity, as detailed further herein, inter alia with reference to FIG. 5.

To illustrate the clustering of the events 155 that are associated with a respective time interval (e.g., 205) into neuronal avalanches in two ways, attention is now drawn to FIGS. 3A and 3B. FIG. 3A is a schematic illustration that illustrates an example of a first way (170-a) for clustering events 155 that are associated with neural signals (e.g., 125-a, 125-b, 125-c, . . . , 125-n) into neuronal avalanches, in accordance with the presently disclosed subject matter. FIG. 3B is a schematic illustration that illustrates an example of a second way (170-b) for clustering events 155 that are associated with the neural signals (e.g., 125-a, 125-b, 125-c, . . . 125-n) into neuronal avalanches, in accordance with the presently disclosed subject matter.

FIG. 3A illustrates one non-limiting example of a division of at least a part of a respective time interval (e.g., 205) that is associated with neural signals (e.g., 125-a, 125-b, 125-c, . . . , 125-n) into first consecutive distinct sub-periods 310 of about equal duration. It is to be noted in this regard that all of the respective time interval (e.g., 205) is divided into the first consecutive distinct sub-periods 310 of about equal duration. FIG. 3A illustrates 16 consecutive distinct sub-periods 310 of about equal duration.

FIG. 3B illustrates another non-limiting example of a division of the at least a part of the respective time interval (e.g., 205) that is associated with the neural signals (e.g., 125-a, 125-b, 125-c, . . . , 125-n) into second consecutive distinct sub-periods 315 of about equal duration. It is to be noted in this regard that all of the respective time interval (e.g., 205) is divided into the second consecutive distinct sub-periods 315 of about equal duration. FIG. 3B illustrates 8 consecutive distinct sub-periods 315 of about equal duration, wherein each distinct sub-period 315 of the second consecutive distinct sub-periods 315 shown in FIG. 3B is of a first duration that is twice a second duration of each distinct sub-period 310 of the first consecutive distinct sub-periods 310 shown in FIG. 3A.

In some cases, the distinct sub-periods (e.g., 310, 315) can be of a duration that is between about 0.001 seconds and 0.2 seconds.

Each neuronal avalanche of the neuronal avalanches is defined as one or more consecutive sub-periods of the distinct sub-periods (e.g., 310, 315) within a respective time interval (e.g., 205) in which one or more events 155 that are associated with the neural signals (e.g., 125-a, 125-b, 125-c, . . . , 125-n) are detected. Each event of the events 155 that is detected in the distinct sub-periods (e.g., 310, 315) is illustrated in FIGS. 3A and 3B by a dot. In FIG. 3A, the third, fourth, sixth, eighth, ninth, tenth and fourteenth distinct sub-periods 310 from the left-hand side of FIG. 3A are distinct sub-periods within the respective time interval (e.g., 205) in which at least one events of the events 155 was detected. Specifically, FIG. 3A illustrates the occurrence of one event 155 in each of the third, fourth, sixth, eighth and tenth distinct sub-periods 310, the occurrence of two events 155 in the ninth distinct sub-period 310, and the occurrence of three events 155 in the fourteenth distinct sub-period 310. In FIG. 3B, the second through fifth and seventh distinct sub-periods 315 from the left-hand side of FIG. 3B are distinct sub-periods within the respective time interval (e.g., 205) in which at least one events of the events 155 was detected. Specifically, FIG. 3B illustrates the occurrence of two events 155 in the second distinct sub-period 315, the occurrence of one event in the third and fourth distinct sub-periods 315, and the occurrence of three events 155 in the fifth and seventh distinct sub-periods 315.

Since each neuronal avalanche of the neuronal avalanches is defined as one or more consecutive sub-periods of the distinct sub-periods (e.g., 310, 315) within the respective time interval (e.g., 205) in which one or more events 155 are detected, in FIG. 3A, the third and fourth distinct sub-periods 310 represent a first neuronal avalanche 320-a, the sixth distinct sub-period 310 represents a second neuronal avalanche 320-b, the eighth through tenth distinct sub-periods 310 represent a third neuronal avalanche 320-c, and the fourteenth distinct sub-period 310 represents a fourth neuronal avalanche 320-d. Moreover, in FIG. 3B, the second through fifth distinct sub-periods 315 represent a fifth neuronal avalanche 320-e and the seventh distinct sub-period 315 represents a sixth neuronal avalanche 320-f.

A size of each avalanche of the avalanches (e.g., 320-a, 320-b, 320-c, 320-d, 320-e, 320-f) is defined by a number of the events 155 that are associated with the respective avalanche. With reference to FIG. 3A, a size of avalanche 320-a is two, a size of avalanche 320-b is one, a size of avalanche 320-c is four, and a size of avalanche 320-d is three. With reference to FIG. 3B, a size of avalanche 320-e is seven and a size of avalanche 320-f is three.

A duration of each avalanche of the avalanches (e.g., 320-a, 320-b, 320-c, or 320-d, 320-e, 320-f) is a number of the distinct sub-periods (e.g., 310, 315) that are associated with the respective avalanche multiplied by a duration of a respective distinct sub-period (e.g., 310, 315) of the distinct sub-periods that is associated with the respective avalanche. With reference to FIG. 3A, assuming that the duration of a respective distinct sub-period (e.g., 310) is one, the duration of avalanche 320-a is two, the duration of avalanches 320-b and 320-d is one, and the duration of avalanche 320-c is three. With reference to FIG. 3B, assuming that the duration of a respective distinct sub-period (e.g., 315) is two, the duration of avalanche 320-e is eight, and the duration of avalanche 320-f is two.

An estimate of a branching parameter of each avalanche of the avalanches (e.g., 320-a, 320-b, 320-c, 320-d, 320-e, 320-f) is calculated based on a variation between a first number of the events 155 in a first distinct sub-period (e.g., 310, 315) associated with the respective avalanche and a second number of the events 155 in a successive distinct sub-period (e.g., 310, 315) that is successive to the first distinct sub-period (e.g., 310, 315). For a respective avalanche (e.g., 320-a, 320-c or 320-e) of the avalanches (e.g., 320-a, 320-b, 320-c, 320-d, 320-e, 320-f) that is associated with two or more distinct sub-periods (e.g., 310, 315), the successive distinct sub-period is a second distinct sub-period (e.g., 310, 315) associated with the respective avalanche. For a respective avalanche (e.g., 320-b, 320-d or 320-f) of the avalanches (e.g., 320-a, 320-b, 320-c, 320-d, 320-e, 320-f) that is associated with a single distinct sub-period (e.g., 310, 315), the successive distinct sub-period is the distinct sub-period (e.g., 310, 315) that follows the respective avalanche.

In some cases, the estimate of the branching parameter of each avalanche of the avalanches (e.g., 320-a, 320-b, 320-c, 320-d, 320-e, 320-f) can be calculated by dividing the second number of the events 155 in a successive distinct sub-period that is associated with the respective avalanche by the first number of the events 155 in the first distinct sub-period that is associated with the respective avalanche. It is to be noted that the estimate of the branching parameter of each avalanche of the avalanches (e.g., 320-a, 320-b, 320-c, 320-d, 320-e, 320-f) can be calculated in additional related ways, as known to one skilled in the art.

To illustrate the calculation of the estimate of the branching parameter of a respective avalanche of the avalanches (e.g., 320-a, 320-b, 320-c, 320-d, 320-e, 320-f) by dividing the second number of the events 155 in a successive distinct sub-period that is associated with the respective avalanche by the first number of the events 155 in the first distinct sub-period that is associated with the respective avalanche, attention is drawn to the neuronal avalanches that are shown in FIGS. 3A and 3B. Turning first to FIG. 3A, the neuronal avalanche 320-a extends over two consecutive distinct sub-periods 310. One event is detected during each of the first distinct sub-period and the successive distinct sub-period of the two consecutive distinct sub-periods 310 associated with neuronal avalanche 320-a. Accordingly, the estimate of the branching parameter for the neuronal avalanche 320-a in accordance with the aforesaid calculation is one.

The neuronal avalanche 320-b extends over a single distinct sub-period 310. Since the successive distinct sub-period 310 that is successive to the single distinct sub-period 310 of the neuronal avalanche 320-b follows the occurrence of the neuronal avalanche 320-b, the estimate of the branching parameter for the neuronal avalanche 320-b in accordance with the aforesaid calculation is zero.

The neuronal avalanche 320-c extends over three consecutive distinct sub-periods 310. One event is detected during the first distinct sub-period of the three consecutive distinct sub-periods 310, and two events are detected during the second distinct sub-period of the three consecutive distinct sub-periods 310, the second distinct sub-period being successive to the first distinct sub-period. According, the estimate of the branching parameter for the neuronal avalanche 320-c in accordance with the aforesaid calculation is two.

The neuronal avalanche 320-d, like the neuronal avalanche 320-b, extends over a single distinct sub-period 310. Accordingly, like the neuronal avalanche 320-b, the estimate of the branching parameter for the neuronal avalanche 320-d in accordance with the aforesaid calculation is zero.

Now turning to FIG. 3B, the neuronal avalanche 320-e extends over four consecutive distinct sub-periods 315. Two events are detected during the first distinct sub-period of the four consecutive distinct sub-periods 315, and one event is detected during the second distinct sub-period of the four consecutive distinct sub-periods 315, the second distinct sub-period being successive to the first distinct sub-period. According, the estimate of the branching parameter for the neuronal avalanche 320-e in accordance with the aforesaid calculation is one-half.

The neuronal avalanche 320-f extends over a single distinct sub-period 315. Since the successive distinct sub-period 315 that is successive to the single distinct sub-period 315 of the neuronal avalanche 320-f follows the occurrence of the neuronal avalanche 320-f, the estimate of the branching parameter for the neuronal avalanche 320-f in accordance with the aforesaid calculation is zero.

An inter-avalanche interval is defined as an interval between consecutive avalanches of the neuronal avalanches (e.g., 320-a, 320-b, 320-c, 320-d, 320-e, 320-f). FIG. 3A illustrates three inter-avalanche intervals (e.g., 330-a, 330-b, 330-c). A duration of each of the inter-avalanche intervals (e.g., 330-a, 330-b, 330-c) is defined by a number of the distinct sub-periods (e.g., 310) that are associated with the respective inter-avalanche interval multiplied by a duration of a respective distinct sub-period (e.g., 310) of the distinct sub-periods. With reference to FIG. 3A, assuming that the duration of a respective distinct sub-period (e.g., 310) is one, the duration of inter-avalanche intervals 330-a and 330-b is one, and the duration of inter-avalanche interval 330-c is three.

FIG. 3B illustrates one inter-avalanche interval (e.g., 330-d). A duration of this inter-avalanche interval (e.g., 330-d) is defined by a number of the distinct sub-periods (e.g., 315) that are associated with the respective inter-avalanche interval multiplied by a duration of a respective distinct sub-period (e.g., 315) of the distinct sub-periods. With reference to FIG. 3B, assuming that the duration of a respective distinct sub-period (e.g., 315) is two, the duration of inter-avalanche interval 330-d is two.

Attention is now drawn to FIG. 4, a block diagram schematically illustrating one example of an abnormal neural activity detection/prediction system 100, in accordance with the presently disclosed subject matter.

In accordance with the presently disclosed subject matter, abnormal neural activity detection/prediction system 100 can optionally comprise a network interface 410 that is configured to connect the abnormal neural activity detection/prediction system 100 to a communications network, through which the abnormal neural activity detection/prediction system 100 can connect to other computerized devices. The network interface 410 can be configured to enable the abnormal neural activity detection/prediction system 100 to send data and receive data sent thereto through the communications network.

Abnormal neural activity detection/prediction system 100 also comprises, or is otherwise associated with, a data repository 420 (e.g. a database, a storage system, a memory including Read Only Memory—ROM, Random Access Memory—RAM, or any other type of memory, etc.) configured to store data. In some cases, the stored data can include at least one given model 425, as detailed further herein, inter alia with reference to FIG. 5. Data repository 420 can be configured to enable retrieval and/or updating and/or deletion of the stored data. It is to be noted that in some cases, data repository 420 can be distributed, while the abnormal neural activity detection/prediction system 100 has access to the information stored thereon, e.g., via a wired or wireless network to which abnormal neural activity detection/prediction system 100 is able to connect (utilizing its network interface 410).

Abnormal neural activity detection/prediction system 100 also comprises a processing circuitry 430. Processing circuitry 430 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant abnormal neural activity detection/prediction system 100 resources and for enabling operations related to abnormal neural activity detection/prediction system 100 resources.

In some cases, processing circuitry 430 can be configured to include a pre-processing module 120. In some cases, processing circuitry 430 can be configured, e.g. using pre-processing module 140, to pre-process neural data (e.g., 115-a, 115-b, 115-c, . . . 115-n) that is obtained from the channels 110, as detailed earlier herein, inter alia with reference to FIG. 1. In some cases, the neural data (e.g., 115-a, 115-b, 115-c, . . . 115-n) can be pre-processed to generate neural signals (e.g., 125-a, 125-b, 125-c, . . . , 125-n), as detailed earlier herein, inter alia with reference to FIG. 1.

In some cases, processing circuitry 430 can be configured to include a feature extraction module 130. In some cases, processing circuitry 430 can be configured, e.g. using feature extraction module 130, to extract, for one or more time intervals, a plurality of features that are associated with the respective time interval, based on neural data (e.g., 115-a, 115-b, 115-c, . . . , 115-n) that is indicative of neural activity in the brain of a monitored person during the respective time interval, as detailed earlier herein, inter alia with reference to FIG. 1, and as detailed further herein, inter alia with reference to FIGS. 5 to 7. In some cases, the plurality of features can include given features that are extracted based on neuronal avalanches, as detailed further herein, inter alia with reference to FIGS. 5 to 7.

Processing circuitry 430 can be configured to include an abnormal neural activity detection/prediction module 140. Processing circuitry 430 can be configured, e.g. using abnormal neural activity detection/prediction module 140, to detect or predict instances of an abnormal neural activity in a brain of a monitored person. In some cases, each instance of the abnormal neural activity can be detected or predicted based on a plurality of features that are associated with a respective time interval, the plurality of features being based on neural data that is associated with the respective time interval. In some cases, the plurality of features can include given features that are extracted based on neuronal avalanches, as detailed further herein, inter alia with reference to FIGS. 5 to 7.

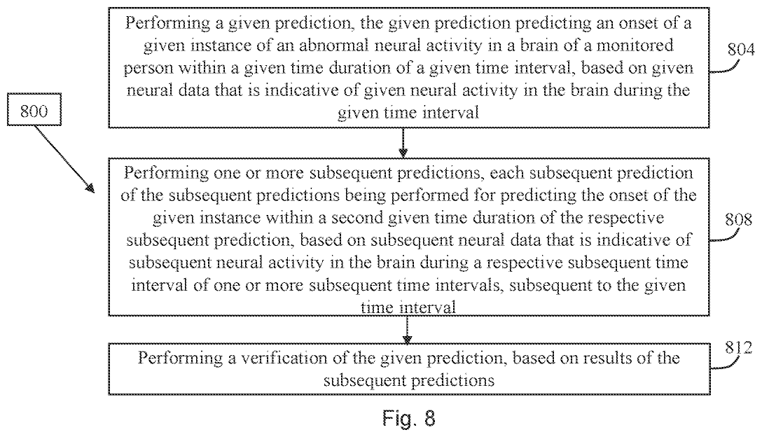

In some cases, processing circuitry 430 can be configured, e.g. using abnormal neural activity detection/prediction module 140, to verify a given prediction predicting an onset of a given instance of an abnormal neural activity in a brain of a monitored person, as detailed further herein, inter alia with reference to FIG. 8. Additionally, or alternatively, in some cases, processing circuitry 430 can be configured, e.g. using abnormal neural activity detection/prediction module 140, to determine, for one or more given time intervals within a given evaluation period, a probability of an onset of an instance of the abnormal neural activity within a given time duration of the respective given time interval, thereby enabling an evaluation of an effectiveness of a treatment plan for the monitored person, as detailed further herein, inter alia with reference to FIG. 10.

In some cases, processing circuitry 430 can be configured to include an events detection module 150. In some cases, processing circuitry 430 can be configured, e.g. using events detection module 150, to detect events 155 that are associated with neural signals (e.g., 125-a, 125-b, 125-c, . . . , 125-n), as detailed earlier herein, inter alia with reference to FIGS. 1 and 2.

In some cases, processing circuitry 430 can be configured to include an events data clustering module 160. In some cases, processing circuitry 430 can be configured, e.g. using events data clustering module 160, to cluster events 155 into neuronal avalanches, as detailed earlier herein, inter alia with reference to FIGS. 1 and 3.

In some cases, processing circuitry 430 can be configured to include an actions performance module 440. In some cases, processing circuitry 430 can be configured, e.g. using actions performance module 440, to automatically perform one or more actions in response to detecting or predicting a given instance of an abnormal neural activity, as detailed further herein, inter alia with reference to FIGS. 5, 8 and 9.

In some cases, processing circuitry 430 can be configured to include a treatment effectiveness module 450. In some cases, processing circuitry 430 can be configured, e.g. using treatment effectiveness module 450, to evaluate an effectiveness of a treatment plan for suppressing an onset of an abnormal neural activity in the brain of the monitored person, as detailed further herein, inter alia with reference to FIG. 10.

Attention is now drawn to FIG. 5, a flow diagram schematically illustrating one example of a sequence of operations 500 for detecting or predicting a given instance of an abnormal neural activity in the brain of a monitored person, in accordance with the presently disclosed subject matter.

In accordance with the presently disclosed subject matter, in some cases, abnormal neural activity detection/prediction system 100 can be configured, e.g. using feature extraction module 130, to extract a plurality of features (e.g., 135-a, . . . , 135-m) based on one or more neural signals (e.g., 125-a, 125-b, 125-c, . . . , 125-n) that are indicative of given neural activity in the brain of the monitored person during a given time interval. The neural signals (e.g., 125-a, 125-b, 125-c, . . . , 125-n) are generated based on neural data (e.g., 115-a, 115-b, 115-c, . . . , 115-n) that is sampled from the plurality of channels 110 during the given time interval, as detailed earlier herein, inter alia with reference to FIG. 1.

In some cases, the plurality of features (e.g., 135-a, . . . , 135-m) that are extracted based on the neural signals (e.g., 125-a, 125-b, 125-c, . . . , 125-n) can include given features that are extracted based on neuronal avalanches (e.g., 320-a, 320-b, 320-c, 320-d, 320-e, 320-f). Each avalanche of the neuronal avalanches (e.g., 320-a, 320-b, 320-c, 320-d, 320-e, 320-f) is one or more consecutive sub-periods of distinct sub-periods (e.g., 310, 315) within the given time interval in which one or more events 155 associated with one or more of the neural signals (e.g., 125-a, 125-b, 125-c, . . . , 125-n) are detected, as detailed earlier herein, inter alia with reference to FIGS. 1 to 3 (block 504).

In some cases, the given features can include one or more inter-avalanche features that are extracted based on durations of inter-avalanche intervals (e.g., 330-a, 330-b, 330-c, 330-d) between consecutive avalanches of the avalanches (e.g., 320-a, 320-b, 320-c, 320-d, 320-e, 320-f).

In some cases, the inter-avalanche features can include one or more distribution-based features that are extracted based on an inter-avalanche distribution of the durations of the inter-avalanche intervals (e.g., 330-a, 330-b, 330-c, 330-d) over the given time interval for a respective division (e.g., 170-a, 170-b) of the given time interval, as detailed further herein, inter alia with reference to FIG. 6. Additionally, or alternatively, in some cases, the inter-avalanche features can include one or more trend-based features that are extracted based on a trend of durations of the inter-avalanche intervals (e.g., 330-a, 330-b, 330-c, 330-d) over at least a part of the given time interval for the respective division (e.g., 170-a, 170-b).

The trend-based features can be extracted by clustering the inter-avalanche intervals (e.g., 330-a, 330-b, 330-c) into a plurality of groups, each group of the groups including consecutive inter-avalanche intervals of the inter-avalanche intervals (e.g., 330-a, 330-b, 330-c). The inter-avalanche intervals can be clustered into one or more of the following groups: pairs, triplets or sequences of consecutive inter-avalanche intervals. In some cases, the trend-based features can be extracted based on a statistical analysis of the groups of inter-avalanche intervals. In some cases, the statistical analysis can be based on higher-order statistics (HOS). In some cases, the trend-based features can be extracted based on second-order or third-order correlations, trends, or gradients.

The distribution-based features of the inter-avalanche features can be extracted based on an inter-avalanche distribution of the durations of the inter-avalanche intervals (e.g., 330-a, 330-b, 330-c) over the given time interval (e.g., 205). In some cases, the inter-avalanche distribution comprises: (a) a first regime for inter-avalanche intervals (e.g., 330-a, 330-b, 330-c) that are of a short duration, the first regime being characterized by a first power law having a first exponent; (b) a second regime for inter-avalanche intervals (e.g., 330-a, 330-b, 330-c) that are of a long duration, longer than the short duration, the second regime being characterized by a second power law having a second exponent, different than the first exponent; and (c) a transition region between the first regime and the second regime.

To explain the extraction of the distribution-based features of the inter-avalanche features, attention is now drawn to FIG. 6, a graph that schematically illustrates one example of an inter-avalanche distribution 600 of durations of inter-avalanche intervals (e.g., 330-a, 330-b, 330-c) over the given time interval (e.g., 205), in accordance with the presently disclosed subject matter. Each of the data points 610 is indicative of a duration of at least one of the inter-avalanche intervals (e.g., 330-a, 330-b, 330-c) over the given time interval (e.g., 205). As illustrated in FIG. 6, the probability that any respective inter-avalanche interval of the inter-avalanche intervals (e.g., 330-a, 330-b, 330-c) over the given time interval (e.g., 205) is of a short duration, being less than about 100 milliseconds in FIG. 6, is greater than the probability that any respective inter-avalanche interval of the inter-avalanche intervals (e.g., 330-a, 330-b, 330-c) over the given time interval (e.g., 205) is of a long duration, being greater than about 100 milliseconds in FIG. 6.

Moreover, as illustrated in FIG. 6, many of the data points 610 that are of a short duration can be fitted to a first power law having a first exponent, the first power law characterizing a first regime 620, and many of the data points 610 that are of a long duration can be fitted to a second power law having a second exponent, different than the first exponent, the second power law characterizing a second regime 630. That is, the inter-avalanche distribution in FIG. 6 comprises the first regime 620 and the second regime 630. The inter-avalanche distribution in FIG. 6 further comprises a transition region 640 between the first regime 620 and the second regime 630 that is associated with inter-avalanche interval durations that are between the durations of the inter-avalanche intervals of a short duration and the durations of the inter-avalanche intervals of a long duration.

In some cases, the distribution-based features of the inter-avalanche features can include at least one of: (i) a first estimate of the first exponent of the first power law that characterizes the first regime (e.g., 620); (ii) a second estimate of the second exponent of the second power law that characterizes the second regime (e.g., 630); (iii) a third estimate of a crossover point between the first regime (e.g., 620) and the second regime (e.g., 630), being an inter-avalanche interval duration within the transition region (e.g., 640); (iv) a first deviation of the inter-avalanche intervals that are of the short duration from the first power law that characterizes the first regime 620; or (v) a second deviation of the inter-avalanche intervals that are of the long duration from the second power law that characterizes the second regime 630. In some cases, the first deviation can be determined based on a goodness of fit of the set of the points 610 that are fitted to the first power law to the first power law. Additionally, or alternatively, in some cases, the second deviation can be determined based on a goodness of fit of the set of the points 610 that are fitted to the second power law to the second power law.

In some cases, the given features that are extracted based on neuronal avalanches (e.g., 320-a, 320-b, 320-c, 320-d, 320-e, 320-f) within the given time interval (e.g., 205) can include one or more multi-scale criticality features. The multi-scale criticality features are extracted by analyzing one or more basic features that are associated with the neuronal avalanches (e.g., 320-a, 320-b, 320-c, 320-d, 320-e, 320-f) for different divisions (e.g., 170-a, 170-b) of the given time interval (e.g., 205) into the distinct sub-periods (e.g., 310, 315). It is to be noted that at least one of the basic features (e.g., all of the basic features) that is analyzed to extract at least one multi-scale criticality feature of the multi-scale criticality features can itself be a given feature of the given features.

In some cases, one of the basic features can be an estimate of a branching parameter for the neuronal avalanches. That is, for each division of two or more different divisions (e.g., 170-a, 170-b) of the given time interval (e.g., 205), the branching parameter for the neuronal avalanches (e.g., 320-a, 320-b, 320-c, 320-d, 320-e, 320-f) that are associated with the respective division can be estimated. The branching parameter for the neuronal avalanches (e.g., 320-a, 320-b, 320-c, 320-d) that are associated with any respective division (e.g., 170-a) of the divisions (e.g., 170-a, 170-b) can be estimated based on an estimate of the branching parameter that is calculated for each of the neuronal avalanches (e.g., 320-a, 320-b, 320-c, 320-d) that are associated with the respective division (e.g., 170-a). The manner in which the estimate of the branching parameter is calculated for any neuronal avalanche of the neuronal avalanches (e.g., 320-a, 320-b, 320-c, 320-d, 320-e, 320-f) is detailed earlier herein, inter alia with reference to FIGS. 3A and 3B. In some cases, the branching parameter for all of the neuronal avalanches (e.g., 320-a, 320-b, 320-c, 320-d) that are associated with a respective division (e.g., 170-a) of the given time interval (e.g., 205) can be estimated by averaging the branching parameter estimates of the respective neuronal avalanches (e.g., 320-a, 320-b, 320-c, 320-d) that are associated with the respective division (e.g., 170-a).

In some cases, a regime of a size distribution of sizes of the avalanches (e.g., 320-a, 320-b, 320-c, 320-d, 320-e, 320-f) over the given time interval (e.g., 205) can be characterized, for two or more different divisions (e.g., 170-a, 170-b) of the given time interval (e.g., 205), by a power law having an exponent. An exponential cut-off of the size distribution is the cut-off transition point between the regime that is well-fit by the power law and the exponential regime at the tail of the size distribution. In some cases, the basic features can include at least one of: (a) an estimate of the exponent of the power law that characterizes the regime of the size distribution, (b) the cut-off transition point, or (c) a deviation of the avalanche sizes that are fitted to the power law that characterizes the regime of the size distribution from the power law. In some cases, the deviation can be determined based on a goodness of fit of the set of points associated with the sizes of the avalanches (e.g., 320-a, 320-b, 320-c, 320-d, 320-e, 320-f) that are fitted to the power law characterizing the regime of the size distribution to the power law.

In some cases, a regime of a duration distribution of durations of the neuronal avalanches (e.g., 320-a, 320-b, 320-c, 320-d, 320-e, 320-f) over the given time interval can be characterized, for two or more different divisions (e.g., 170-a, 170-b) of the given time interval (e.g., 205), by a power law having an exponent. In some cases, one of the basic features can be an estimate of the exponent of the power law that characterizes the regime of the duration distribution.

In some cases, for a plurality of durations of the neuronal avalanches (e.g., 320-a, 320-b, 320-c, 320-d, 320-e, 320-f) over the given time interval for a respective division of two or more different divisions (e.g., 170-a, 170-b) of the given time interval (e.g., 205), a power law relationship indicates a dependence of a mean size of the avalanches for each duration of the durations on the respective duration, the power law relationship having an exponent. In some cases, one of the basic features can be an estimate of the exponent of the power law relationship.

In some cases, the inter-avalanche distribution of the durations of the inter-avalanche intervals (e.g., 330-a, 330-b, 330-c, 330-d) over the given time interval (e.g., 205) can comprise, for two or more different divisions (e.g., 170-a, 170-b) of the given time interval (e.g., 205), (a) a first regime for inter-avalanche intervals that are of a short duration, the first regime being characterized by a first power law having a first exponent; (b) a second regime for inter-avalanche intervals that are of a long duration, longer than the short duration, the second regime being characterized by a second power law having a second exponent, different than the first exponent; and (c) a transition region between the first regime and the second regime. In some cases, the basic features can include at least one: a first estimate of the first exponent, a second estimate of the second exponent, or a third estimate of a crossover point between the first regime and the second regime, being an inter-avalanche interval duration within the transition region.

In some cases, for one or more of the basic features, the multi-scale criticality features can include an offset and a slope for a linear model that indicates a dependence of the respective basic feature on two or more different divisions (e.g., 170-a, 170-b) of the given time interval (e.g., 205).

Additionally, or alternatively, in some cases, for one or more pairs of the basic features including a first basic feature and a second basic feature, the multi-scale criticality features can include an offset and a slope for a linear model that indicates a dependence of a relationship between the first basic feature and the second basic feature (e.g., a relationship between the size distribution and the duration distribution of the neuronal avalanches) of the respective pair on two or more different divisions (e.g., 170-a, 170-b) of the given time interval (e.g., 205).

The neuronal avalanches consist of main avalanches and secondary avalanches, the main avalanches being the neuronal avalanches of a first size that is greater than or equal to a size threshold. The size threshold can be, for example (but not limited to), 15, 20, 25, 30, 35 or 40 events. In some cases, the given features that are based on the neuronal avalanches (e.g., 320-a, 320-b, 320-c, 320-d, 320-e, 320-f) can include one or more avalanche features that are extracted by analyzing a secondary avalanche rate distribution representing rates of secondary avalanches as a function of time that has elapsed since a preceding main avalanche of the main avalanches immediately preceding the secondary avalanches. In some cases, the secondary avalanche rate distribution can include a regime that is characterized by a power law having an exponent.

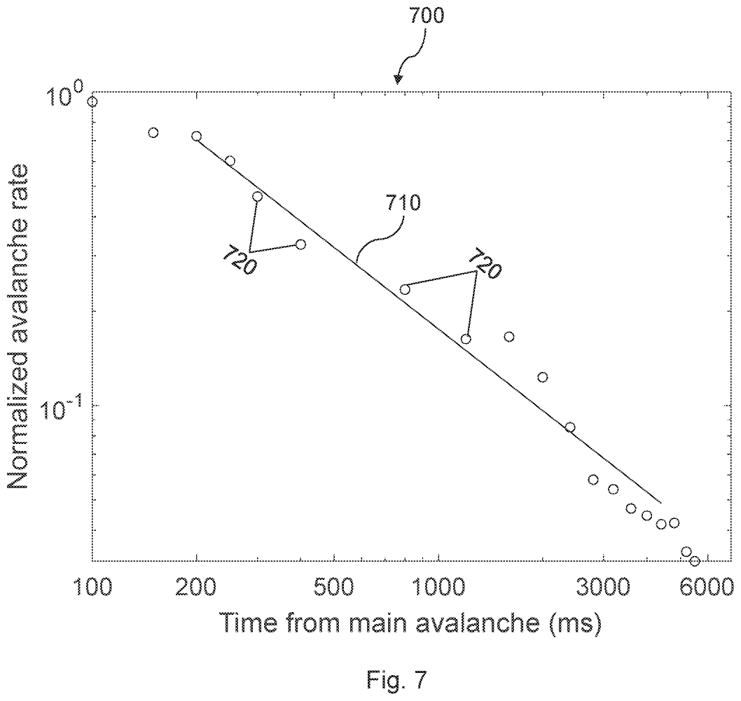

To illustrate this, attention is now drawn to FIG. 7, a graph that schematically illustrates one example of a secondary avalanche rate distribution 700 of a normalized secondary avalanche rate as a function of time that has elapsed since a preceding main avalanche, in accordance with the presently disclosed subject matter.

In accordance with the presently disclosed subject matter, in some cases, the secondary avalanche rate distribution 700 includes a regime 710 that is characterized by a power law having an exponent, wherein the rate of secondary avalanches generally decreases as a function of time since a preceding main avalanche. The exponent of the power law is determined in accordance with the data points 720 that are indicative of the values of the rates of secondary avalanches as a function of time that has elapsed since a preceding main avalanche.

In some cases, the avalanche features that are extracted by analyzing a secondary avalanche rate distribution can include at least one of: (a) an estimate of the exponent of the power law that characterizes the regime (e.g., 710) of the secondary avalanche rate distribution or (b) a deviation of the (normalized) rates of the secondary avalanches that are fitted to the power law that characterizes the regime (e.g., 710) from the power law. In some cases, this deviation can be determined by performing a goodness of fit that is indicative of how well the regime (e.g., 710) fits the data points (e.g., 720).

In some cases, the given features that are based on the neuronal avalanches (e.g., 320-a, 320-b, 320-c, 320-d, 320-e, 320-f) can include one or more additional avalanche features. In order to extract the additional avalanche features, for each pair of consecutive avalanches over the given time interval (e.g., 205), both the duration of the inter-avalanche interval between the consecutive avalanches and the difference in size between the consecutive avalanches (the difference in size between the later avalanche of the consecutive avalanches and the earlier avalanche of the consecutive avalanches, or alternatively, the difference in size between the earlier avalanche of the consecutive avalanches and the later avalanche of the consecutive avalanches) are calculated. Based on this data, a function for the given time interval (e.g., 205) can be generated that estimates a relation between: (a) a difference in a size between consecutive avalanches and (b) a duration of an inter-avalanche interval between the consecutive avalanches.

In some cases, the additional avalanche features can include a value of a duration of the inter-avalanche interval at which a result of the function transitions from a negative value to a positive value or vice versa. Additionally, or alternatively, in some cases, at least one of the additional avalanche features can be extracted by analyzing a shape of the function.

It is to be noted that the plurality of the extracted features (e.g., 135-a, . . . , 135-m) can include any combination of the given features detailed above.

Returning to FIG. 5, abnormal neural activity detection/prediction system 100 can be configured, e.g. using abnormal neural activity detection/prediction module 140, to detect a given instance of an abnormal neural activity in the brain of the monitored person or to predict the given instance within a given time duration of the given time interval (e.g., 205). The given instance can be detected or predicted in accordance with the plurality of features (e.g., 135-a, . . . , 135-m) that are extracted based on the neural signals (125-a, 125-b, 125-c, . . . , 125-n) that are associated with the given time interval (e.g., 205), including, in some cases, the given features that are extracted based on the neuronal avalanches (block 508). In some cases, the given instance can be detected or predicted also in accordance with other features that are extracted based on the neural data (e.g., 115-a, 115-b, 115-c, . . . , 115-n) but not necessarily the neural signals (125-a, 125-b, 125-c, . . . , 125-n). Additionally, or alternatively, in some cases, the given instance can be detected or predicted based also on one or more additional vital signs of the monitored person, for example, but not limited to, movement by the monitored person, audio information provided by the monitored person, heart rate variability for the monitored person, electrodermal activity for the monitored person, eye movements of the monitored person, or pupil size of an eye of the monitored person.

In some cases, the features that are extracted based on the neural data (e.g., 115-a, 115-b, 115-c, . . . , 115-n), e.g., based on the neural signals (125-a, 125-b, 125-c, . . . , 125-n), can be analyzed by at least one given model 425 to detect or predict the given instance of the abnormal neural activity. In some cases, abnormal neural activity detection/prediction system 100 can be configured to detect a given instance of an abnormal neural activity in the brain of the monitored person based on a determination by the given model 425 that a probability that the given instance of the abnormal neural activity has been detected is greater than a threshold probability. Additionally, or alternatively, in some cases, abnormal neural activity detection/prediction system 100 can be configured to predict a given instance of an abnormal neural activity in the brain of the monitored person within a given time duration of a given time interval (e.g., 205) upon a determination by the given model 425 that a probability of the onset of the given instance of the abnormal neural activity within the given time duration is greater than a threshold probability.

In some cases, the given model 425 can be a Machine Learning (ML) or Deep Learning (DL) model. The ML or DL model can be trained using one or more ML or DL algorithms, for example: logistic regression, Linear Discriminant Analysis (LDA), Support-Vector Machine (SVM), light Gradient Boosting Machines (light GBM), random forest, extra trees, or deep neural networks. Additionally, or alternatively, in some cases, the given model 425 can be a regression model. In some cases, the regression model can be a linear regression model, for example a linear regression model that has been fit using a least squares approach. It is to be noted that for the purposes of this disclosure, the given model 425 can be any model that is configured to analyze the plurality of features (e.g., 135-a, . . . , 135-m) to detect or predict the given instance of the abnormal neural activity.

The given model 425 can be trained or fit based on a plurality of historical records. In some cases, abnormal neural activity detection/prediction system 100 can be configured to generate the given model 425 by training or fitting the given model 425 based on the plurality of historical records. Each historical record of the historical records includes the plurality of features that are extracted to detect or predict the given instance of the abnormal neural activity in the brain of the monitored person, wherein the features are extracted for each of the historical records based on historical neural signals that are indicative of historical neural data in the brain of a respective person during a historical time interval that precedes the given time interval (e.g., 205). Moreover, each historical record of the historical records further includes at least one label that is indicative of at least one of the following: whether a respective instance of the abnormal neural activity occurred during the respective time interval associated with the respective historical record or whether the respective instance was predicted within the given time duration of the respective time interval associated with the respective historical record. In this manner, the given model 425 can be configured to analyze the extracted features associated with the monitored person to perform detection and/or prediction of given instances of the abnormal neural activity in the brain of the monitored person.

In some cases, the given model 425 can be trained or fit based on a plurality of historical records that are associated with a group of persons that are characterized by at least one attribute that is common to all of the members of the group and to the monitored person, for example, a group of persons within a given age range. In some cases, one or more of the historical records are associated with the monitored person.

In some cases, the given model 425 can be personalized for the monitored person by training or fitting the given model 425 based only on historical records that are associated with the monitored person.

In some cases, for at least some of the historical records that are associated with the detection or prediction of a respective instance of the abnormal neural activity, the respective historical record can provide one or more characteristics that are associated with the respective instance that is detected or predicted by the respective historical record. In this manner, the given model 425 can provide these characteristics for a given instance of the abnormal neural activity that is detected or predicted for the monitored person.

In some cases, the abnormal neural activity can be an epileptic seizure. In some cases, one or more characteristics that are provided by the given model 425 can include at least one of: a type of the epileptic seizure, one or more spatial characteristics associated with the epileptic seizure, a duration of the epileptic seizure, or a sub-duration within the given time duration of the given time interval (e.g., 205) in which the epileptic seizure is expected to occur. The types of the epileptic seizure that can be predicted can include, for example, one or more of: a focal epileptic seizure, a central epileptic seizure, or a nocturnal epileptic seizure. Moreover, the spatial characteristics that are associated with the epileptic seizure can include, for example, one or more of: (a) locations of the channels 100 that are associated with the events that are detected during the given time interval (e.g., 205), and based on which the given features of the plurality of features (e.g., 135-a, . . . , 135-m) that are used to detect or predict the given instance of the epileptic seizure are extracted, or (b) the number of the channels 110 that are associated with the events.

In some cases, the given instance of the abnormal neural activity in the brain of the monitored person can be detected or predicted without using a given model 425. For example, the given instance can be detected or predicted by direct thresholding the features (135-a, . . . , 135-m) that are extracted for the monitored person, e.g., based on historical data. It is to be noted that the present disclosure is intended to cover any manner of detecting or predicting the given instance of the abnormal neural activity, based on the features (e.g., 135-a, . . . , 135-m) that are extracted for the monitored person.

In some cases, abnormal neural activity detection/prediction system 100 can be configured to generate a new training record based on the plurality of features (e.g., 135-a, . . . , 135-m) that are extracted for a given time interval (e.g., 205) and whether a given instance of the abnormal neural activity occurred during the given time interval (e.g., 205) or within a given time duration of the given time interval (e.g., 205). The new training record can include the plurality of features (e.g., 135-a, . . . , 135-m) and at least one label, the label being indicative of whether the given instance occurred during the given time interval (e.g., 205) or within the given time duration of the given time interval (e.g., 205).

Moreover, in some cases, abnormal neural activity detection/prediction system 100 can be configured to update a set of training records for training one or more models to include the new training record, giving rise to an updated set of training records for the one or more models. In some cases, the one or more models can include the given model 425. In addition, abnormal neural activity detection/prediction system 100 can be configured to update the one or more models based on the updated set of training records.

Returning to FIG. 5, in some cases, in response to detecting or predicting a given instance of an abnormal neural activity in the brain of the monitored person, abnormal neural activity detection/prediction system 100 can be configured, e.g. using actions performance module 440, to automatically perform one or more actions (block 512).

In some cases, the actions can include providing an alert. In some cases, the alert can be provided to the monitored person and/or to a caregiver (e.g., a medical practitioner) of the monitored person. In some cases, the alert can be sent to a remote computing entity, remote from the abnormal neural activity detection/prediction system 100. The alert can be provided in any manner, for example, using at least one of: audio, video, or vibration.

In some cases, the alert is provided only upon multiple predictions of the given instance of the abnormal neural activity, as detailed further herein, inter alia with reference to FIG. 8.

In some cases, following a prediction of a given instance of the abnormal neural activity, the actions can include providing both: (a) an alert and (b) a confidence level that is indicative of a probability of the onset of the given instance within the given time duration of the given time interval, as detailed further herein, inter alia with reference to FIG. 8.

In some cases, the actions can include automatically assisting the monitored person, for example by providing a treatment plan for the monitored person, as detailed further herein, inter alia with reference to FIG. 9.

Attention is now drawn to FIG. 8, a flow diagram schematically illustrating one example of a sequence of operations 800 for verifying a given prediction predicting an onset of a given instance of an abnormal neural activity in a brain of a monitored person, in accordance with the presently disclosed subject matter.

In accordance with the presently disclosed subject matter, abnormal neural activity detection/prediction system 100 can be configured, e.g. using abnormal neural activity detection/prediction module 140, to perform the given prediction, the given prediction predicting the onset of the given instance of the abnormal neural activity within a given time duration of a given time interval. The given prediction is based on given neural data that is indicative of given neural activity in the brain of the monitored person during the given time interval, as detailed earlier herein, inter alia with reference to FIGS. 1 to 5 (block 804). In some cases, the given prediction can also provide one or more characteristics associated with the predicted abnormal neural activity, for example, an epileptic seizure, as detailed earlier herein, inter alia with reference to FIG. 5.

Following the given prediction, abnormal neural activity detection/prediction system 100 can be configured, e.g. using abnormal neural activity detection/prediction module 140, to perform one or more subsequent predictions, each subsequent prediction of the subsequent predictions being performed for predicting the onset of the given instance of the abnormal neural activity within a second given time duration of the respective subsequent prediction. Each subsequent prediction of the subsequent predictions predicts the onset of the given instance based on subsequent neural data that is indicative of subsequent neural activity in the brain of the monitored person during a respective subsequent time interval of one or more subsequent time intervals, subsequent to the given time interval (block 808). In some cases, the second given time duration is identical to the given time duration. In some cases, the second given time duration is different than the given time duration, e.g., less than the given time duration. In some cases, the second given time duration can be within a range of 5 minutes to 8 hours. In some cases, the second given time duration can be within a range of 10 minutes to 4 hours (e.g., 10 minutes, 15 minutes, 20 minutes, 25 minutes, 30 minutes, 35 minutes, 40 minutes, 45 minutes, 50 minutes, 55 minutes, one hour, 75 minutes, 90 minutes, 105 minutes, two hours, two and a half hours, three hours, three and a half hours, four hours). The subsequent predictions are based on the same features as the given prediction. In some cases, the subsequent time intervals are (immediately) successive to the given time interval.

Abnormal neural activity detection/prediction system 100 can be further configured, e.g. using abnormal neural activity detection/prediction module 140, to perform a verification of the given prediction, based on results of the subsequent predictions, as detailed below (block 812).

The verification of the given prediction can be performed in one or more ways. In some cases, the given prediction can be verified upon one or more of the subsequent predictions predicting the onset of the given instance of the abnormal neural activity within the second given time duration of the respective subsequent prediction. Alternatively, in some cases, the given prediction can be verified upon at least a given percentage of the subsequent predictions predicting the onset of the given instance within the second given time duration of the respective subsequent prediction. Additionally, or alternatively, in some cases, the given prediction can be verified upon at least two successive predictions of the subsequent predictions predicting the onset of the given instance within the second given time duration of the respective successive prediction, wherein the successive predictions are performed for successive time intervals of the subsequent time intervals. Additionally, or as a further alternative, in some cases, the given prediction can be verified upon at least one subsequent prediction of the subsequent predictions that is performed for at least one successive time interval of the subsequent time intervals that is successive to the given time interval predicting the onset of the given instance within the second given time duration of the respective subsequent prediction.

In some cases, in response to verifying the given prediction, abnormal neural activity detection/prediction system 100 can be configured, e.g. using actions performance module 440, to perform one or more actions.

In some cases, the actions can include providing an alert. Moreover, in some cases, the actions can further include providing a confidence level, the confidence level being indicative of a probability of the onset of the given instance, for example, within the given time duration of the given time interval, and being based on a result of at least one of the subsequent predictions (e.g., the last subsequent prediction of the subsequent predictions, the last two subsequent predictions of the subsequent predictions, all of the subsequent predictions, the given prediction and the subsequent predictions, etc.). Additionally, or alternatively, in some cases, the actions can include automatically assisting the monitored person, for example by providing a treatment plan for the monitored person, as detailed further herein, inter alia with reference to FIG. 9.

In some cases, in response to verifying the given prediction, abnormal neural activity detection/prediction system 100 can be configured, e.g. using abnormal neural activity detection/prediction module 140, to perform one or more additional predictions, each additional prediction of the additional predictions being performed for predicting the onset of the given instance within a third given time duration of the respective additional prediction, based on additional neural data that is indicative of additional neural activity in the brain of the monitored person during a respective additional time interval subsequent to the subsequent time intervals. The additional predictions are based on the same features as the given prediction. In some cases, the third given time duration is identical to the given time duration. In some cases, the third given time duration is different than the given time duration, e.g., less than the given time duration. In some cases, the third given time duration can be within a range of 5 minutes to 8 hours. In some cases, the third given time duration can be within a range of 10 minutes to 4 hours (e.g., 10 minutes, 15 minutes, 20 minutes, 25 minutes, 30 minutes, 35 minutes, 40 minutes, 45 minutes, 50 minutes, 55 minutes, one hour, 75 minutes, 90 minutes, 105 minutes, two hours, two and a half hours, three hours, three and a half hours, four hours).

Abnormal neural activity detection/prediction system 100 can be further configured, e.g. using abnormal neural activity detection/prediction module 140, to update the confidence level that is provided in response to verifying the given prediction, based on the additional predictions. In some cases, this can enable improving an accuracy of the detection of the given instance of the abnormal neural activity.

In some cases, in which the updated confidence level indicates that the alert was improperly provided (i.e., the prediction of the onset of the given instance is no longer valid), abnormal neural activity detection/prediction system 100 can be configured, e.g. using actions performance module 440, to cancel the alert.

In some cases, responsive to the given prediction, abnormal neural activity detection/prediction system 100 can be configured, e.g. using actions performance module 440, to provide both: (a) an alert and (b) a confidence level that is indicative of a result of the given prediction. In some cases, as noted earlier herein, the given prediction provides a respective probability of the onset of the given instance within the given time duration of the given time interval. In such cases, the confidence level can be the respective probability.

In some cases, abnormal neural activity detection/prediction system 100 can be configured, e.g. using abnormal neural activity detection/prediction module 140, to update the confidence level, based on the subsequent predictions, subsequent to the given prediction. In some cases, the verification of the given prediction can be based on the updated confidence level being indicative of an expected onset of the given instance of the abnormal neural activity. In some cases, the verification of the given prediction can enable improving an accuracy of the detection of the given instance of the abnormal neural activity. In some cases, in which the given prediction is unverified based on the updated confidence level (i.e., the updated confidence level indicates that the prediction of the onset of the given instance is no longer valid), abnormal neural activity detection/prediction system 100 can be configured, e.g. using actions performance module 440, to cancel the alert.

Attention is now drawn to FIG. 9, a flow diagram schematically illustrating one example of a sequence of operations 900 for automatically assisting a monitored person in response to a detection or prediction of a given instance of an abnormal neural activity in the brain of the monitored person, in accordance with the presently disclosed subject matter.

In accordance with the presently disclosed subject matter, abnormal neural activity detection/prediction system 100 can be configured, e.g. using abnormal neural activity detection/prediction module 140, to detect the given instance or predict, in a given prediction, an onset of the given instance within a given time duration of a given time interval, based on given neural data that is indicative of given neural activity in the brain during the given time interval, as detailed earlier herein, inter alia with reference to FIGS. 1 to 7 (block 904).

Abnormal neural activity detection/prediction system 100 can be further configured, e.g. using actions performance module 440, to automatically assist the monitored person, in response to the detecting of the given instance or the predicting of the onset of the given instance (block 908).

In some cases, automatically assisting the monitored person includes waking the monitored person or guiding the monitored person to a location that is more suitable for the monitored person to undergo the given instance of the abnormal neural activity.

In some cases, automatically assisting the monitored person includes providing a treatment plan for the monitored person for suppressing the given instance or subsequent instances of the abnormal neural activity, the subsequent instances being subsequent to the given instance. In some cases, the treatment plan can include a new drug treatment (e.g., a sedative) or an optimization of an existing drug treatment for the monitored person. Additionally, or alternatively, in some cases, the treatment plan can include vagal stimulation to be performed on the monitored person. Additionally, or as a further alternative, in some cases, the treatment plan can include at least one biofeedback session and/or at least one neurofeedback session for the monitored person. The neurofeedback session can include, for example, meditation and/or viewing animation scenes.

In some cases, abnormal neural activity detection/prediction system 100 can be configured, based on one or more given features that are extracted based on neuronal avalanches at a given time, for example, as detailed earlier herein, inter alia with reference to FIGS. 1 to 7, to perform at least one neurofeedback session that is configured to modulate at least one of the given features at a later time, subsequent to the given time. In this manner, an instance of the abnormal neural activity can be suppressed, regardless of whether the given features extracted based on the neuronal avalanches were used to detect or predict a given instance of the abnormal neural activity.

Attention is now drawn to FIG. 10, a flow diagram schematically illustrating one example of a sequence of operations 1000 for evaluating, during a given evaluation period, an effectiveness of a treatment plan for suppressing an onset of an abnormal neural activity in a brain of a monitored person, the given evaluation period being concurrent with or subsequent to a performance of the treatment plan, in accordance with the presently disclosed subject matter.

In accordance with the presently disclosed subject matter, abnormal neural activity detection/prediction system 100 can be configured, e.g. using abnormal neural activity detection/prediction module 140, to determine, for one or more given time intervals within the given evaluation period, a probability of an onset of an instance of the abnormal neural activity within a given time duration of the respective given time interval. For each of the given time intervals within the given evaluation period, the probability of the onset of the instance of the abnormal neural activity within the given time duration of the respective given time interval is based on given neural data that is indicative of given neural activity in the brain of the monitored person during the respective given time interval, as detailed earlier herein, inter alia with reference to FIGS. 1 to 7 (block 1004).

In some cases, the given neural data includes neural signals that are indicative of the given neural activity, as detailed earlier herein, inter alia with reference to FIGS. 1 to 5.

Abnormal neural activity detection/prediction system 100 can be further configured, e.g. using treatment effectiveness module 450, to evaluate the effectiveness of the treatment plan, based on at least selected probabilities of the determined probabilities for the given time intervals (block 1008). In some cases, the selected probabilities are all of the determined probabilities. Examples of the treatment plan are detailed earlier herein, inter alia with reference to FIG. 9.

In some cases, abnormal neural activity detection/prediction system 100 can be configured, e.g. using abnormal neural activity detection/prediction module 140, to calculate a prediction score, based on the selected probabilities. The prediction score can be calculated, for example, based on one or more of the following: an average of the selected probabilities, a percentage of the selected probabilities that are above a threshold probability, a statistical deviation (e.g., a standard deviation) of the selected probabilities, etc. It is to be noted that for the purposes of the present disclosure, the prediction score can be calculated in any manner that allows for evaluating the effectiveness of the treatment plan for the monitored person. Abnormal neural activity detection/prediction system 100 can be configured to evaluate the effectiveness of the treatment plan based on the prediction score.

In some cases, abnormal neural activity detection/prediction system 100 can be configured, e.g. using abnormal neural activity detection/prediction module 140, to determine, for one or more second given time intervals within an earlier evaluation period preceding the performance of the treatment plan, a second probability of an onset of an earlier instance of the abnormal neural activity within a second given time duration of the respective second given time interval, based on earlier neural data that is indicative of earlier neural activity in the brain of the monitored person during the respective second predefined time interval. In some cases, the second given time duration can be substantially identical to the given time duration. Moreover, based on at least selected second probabilities of the determined second probabilities, abnormal neural activity detection/prediction system 100 can be configured, e.g. using abnormal neural activity detection/prediction module 140, to calculate a second prediction score. In some cases, the selected second probabilities are all of the determined second probabilities. In some cases, the second prediction score can be calculated in the same manner as the prediction score, for example, based on one or more of the following: an average of the selected second probabilities, a percentage of the selected second probabilities that are above a threshold probability, a statistical deviation (e.g., a standard deviation) of the selected second probabilities, etc. Based on the prediction score and the second prediction score, abnormal neural activity detection/prediction system 100 can be configured to evaluate the effectiveness of the treatment plan. As a non-limiting example, if the prediction score is less than the second prediction score or less than the second prediction score by at least a given amount, the treatment plan can be determined to be effective, whereas if the prediction score is greater than the second prediction score or less than the second prediction score by less than the given amount, the treatment plan can be determined to be ineffective.

In some cases, abnormal neural activity detection/prediction system 100 can be configured, e.g. using treatment effectiveness module 450, to evaluate the effectiveness of the treatment plan, based on changes in one or more features that are based on neural activity in the brain of the monitored person and that are extracted during the course of the treatment plan, for example, given features that are extracted based on neuronal avalanches.

It is to be noted that, with reference to FIGS. 5 and 8 to 10, some of the blocks can be integrated into a consolidated block or can be broken down to a few blocks and/or other blocks may be added. It is to be further noted that some of the blocks are optional. It should be also noted that whilst the flow diagram is described also with reference to the system elements that realize them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A method for detecting or predicting a given instance of an abnormal neural activity in a brain of a monitored person, the method comprising:
extracting a plurality of features based on one or more neural signals that are indicative of given neural activity in the brain during a given time interval, wherein given features of the features are extracted based on neuronal avalanches, each avalanche of the avalanches being one or more consecutive sub-periods of distinct sub-periods within the given time interval in which one or more events associated with one or more of the neural signals are detected; and
detecting the given instance or predicting the given instance within a given time duration of the given time interval, based on the plurality of features.

2. The method of claim 1, wherein one or more of the events are associated with a peak amplitude in a respective neural signal of the neural signals that is greater than or equal to a threshold.

3. The method of claim 1, wherein the given features include one or more inter-avalanche features that are extracted based on durations of inter-avalanche intervals between consecutive avalanches of the avalanches.

4. (canceled)

5. (canceled)

6. The method of claim 1, wherein the given features include one or more multi-scale criticality features that are extracted by analyzing one or more basic features that are associated with the avalanches for different divisions of the given time interval into the distinct sub-periods.

7. The method of claim 6, wherein, for one or more of the basic features, the multi-scale criticality features include an offset and a slope for a linear model that indicates a dependence of the respective basic feature on the different divisions.

8. The method of claim 6, wherein, for one or more pairs of the basic features including a first basic feature and a second basic feature, the multi-scale criticality features include an offset and a slope for a linear model that indicates a dependence of a relationship between the first basic feature and the second basic feature of the respective pair on the different divisions.

9. (canceled)

10. (canceled)

11. (canceled)

12. (canceled)

13. (canceled)

14. The method of claim 1, wherein a respective size of each avalanche of the avalanches is defined by a number of the events that are associated with the respective avalanche;
wherein the avalanches consist of main avalanches and secondary avalanches, the main avalanches being the avalanches of a first size greater than or equal to a size threshold; and
wherein the given features include one or more avalanche features that are extracted by analyzing a secondary avalanche rate distribution representing rates of secondary avalanches as a function of time that has elapsed since a preceding main avalanche of the main avalanches immediately preceding the secondary avalanches.

15. The method of claim 14, wherein the secondary avalanche rate distribution includes a regime that is characterized by a power law having an exponent, and wherein the avalanche features include at least one of:
an estimate of the exponent; or
a deviation of the rates of the secondary avalanches fitted to the power law from the power law.

16. The method of claim 1, wherein a respective size of each avalanche of the avalanches is defined by a number of the events that are associated with the respective avalanche; and
wherein the given features include one or more additional avalanche features that are extracted by analyzing a function that estimates a relation between: (a) a difference in a size between consecutive avalanches of the avalanches and (b) a duration of an inter-avalanche interval between the consecutive avalanches.

17. (canceled)

18. (canceled)

19. The method of claim 1, wherein, in response to detecting or predicting the given instance, the method further comprises:
automatically performing one or more actions.

20. (canceled)

21. The method of claim 19, wherein the actions include providing a treatment plan for the monitored person for suppressing the given instance or subsequent instances of the abnormal neural activity, the subsequent instances being subsequent to the given instance.

22. The method of claim 1, wherein the abnormal neural activity is an epileptic seizure.

23. (canceled)

24. (canceled)

25. (canceled)

26. (canceled)

27. (canceled)

28. (canceled)

29.-62. (canceled)

63. A system for detecting or predicting a given instance of an abnormal neural activity in a brain of a monitored person, the system comprising a processing circuitry configured to:
extract a plurality of features based on one or more neural signals that are indicative of given neural activity in the brain during a given time interval, wherein given features of the features are extracted based on neuronal avalanches, each avalanche of the avalanches being one or more consecutive sub-periods of distinct sub-periods within the given time interval in which one or more events associated with one or more of the neural signals are detected; and
detect the given instance or predict the given instance within a given time duration of the given time interval, based on the plurality of features.

64. The system of claim 63, wherein one or more of the events are associated with a peak amplitude in a respective neural signal of the neural signals that is greater than or equal to a threshold.

65. The system of claim 63, wherein the given features include one or more inter-avalanche features that are extracted based on durations of inter-avalanche intervals between consecutive avalanches of the avalanches.

66. (canceled)

67. (canceled)

68. The system of claim 63, wherein the given features include one or more multi-scale criticality features that are extracted by analyzing one or more basic features that are associated with the avalanches for different divisions of the given time interval into the distinct sub-periods.

69. (canceled)

70. (canceled)

71. (canceled)

72. (canceled)

73. (canceled)

74. (canceled)

75. (canceled)

76. The system of claim 63, wherein a respective size of each avalanche of the avalanches is defined by a number of the events that are associated with the respective avalanche;
wherein the avalanches consist of main avalanches and secondary avalanches, the main avalanches being the avalanches of a first size greater than or equal to a size threshold; and
wherein the given features include one or more avalanche features that are extracted by analyzing a secondary avalanche rate distribution representing rates of secondary avalanches as a function of time that has elapsed since a preceding main avalanche of the main avalanches immediately preceding the secondary avalanches.

77. The system of claim 76, wherein the secondary avalanche rate distribution includes a regime that is characterized by a power law having an exponent, and wherein the avalanche features include at least one of:
an estimate of the exponent; or
a deviation of the rates of the secondary avalanches fitted to the power law from the power law.

78. The system of claim 63, wherein a respective size of each avalanche of the avalanches is defined by a number of the events that are associated with the respective avalanche; and
wherein the given features include one or more additional avalanche features that are extracted by analyzing a function that estimates a relation between: (a) a difference in a size between consecutive avalanches of the avalanches and (b) a duration of an inter-avalanche interval between the consecutive avalanches.

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91.-124. (canceled)

125. A non-transitory computer readable storage medium having computer readable program code embodied therewith, the computer readable program code, executable by processing circuitry of a computer to perform a method for detecting or predicting a given instance of an abnormal neural activity in a brain of a monitored person, the method comprising:
extracting a plurality of features based on one or more neural signals that are indicative of given neural activity in the brain during a given time interval, wherein given features of the features are extracted based on neuronal avalanches, each avalanche of the avalanches being one or more consecutive sub-periods of distinct sub-periods within the given time interval in which one or more events associated with one or more of the neural signals are detected; and
detecting the given instance or predicting the given instance within a given time duration of the given time interval, based on the plurality of features.

126.-128. (canceled)
